(19) United States
(12) Patent Application Publication
(10) Pub. No.: US 2003/0022258 A1
(43) Pub. Date: Jan. 30, 2003

(54) Human actin-binding regulatory proteins and methods for detection, diagnosis and treatment of different stages of carcinogenesis

(75) Inventor: Joel D. Pardee, Patterson, NY (US)

Correspondence Address: Irving N. Feit, Esq., HOFFMANN & BARON, LLP, 6900 Jericho Turnpike, Syosset, NY, 11791, US

(73) Assignee: Cornell Research Foundation, Inc.

(21) Appl. No.: 10/105,708

(22) Filed: Mar. 25, 2002

Related U.S. Application Data
(62) Division of application No. 09/586,629, filed on Jun. 5, 2000, now abandoned.
Application No. 09/586,629 is a continuation-in-part of application No. 09/419,485, filed on Oct. 15, 1999, now Pat. No. 6,403,766.

(51) Int. Cl. G01N 33/574; C07H 21/04; C12N 9/02; C12P 21/02; C12N 5/06

(52) U.S. Cl. 435/7.23; 435/69.3; 435/189; 435/320.1; 435/325; 530/388.26; 536/23.2

(57) ABSTRACT

Compositions and methods are provided for actin regulatory proteins including human severin (H-severin) and H-30; and their uses including the preparation of polyclonal and monoclonal antibodies for use in diagnosing and staging the progression of metastatic tumors and other disorders of cellular growth regulation. Also provided are methods of screening to identify potential drug candidate molecules which modulate the activity of human severin or H-30 and methods of use of such compounds to accelerate wound healing, or to treat a metastasis or growth disorder.

### RELATED APPLICATION

This application is a continuation-in-part of U.S. Ser. No. 09/419,485 filed on Oct. 15, 1999 the entire text of which is hereby incorporated by reference.

This work was supported by one or more of the following grants: GM32458 from the N.I.H.; NIH GMS R01-32458; NIH SCOR Grant RFA NIH-95-HL-06-H; and Biomedical Support Grant 507-RR 05396. The government has certain rights to this invention.

### FIELD OF THE INVENTION

This invention relates to the field of control of cytoskeletal structure and changes in the cytoskeletal structure, especially as it relates to the regulation of cell motility, transformation and tumorigenesis. Specifically the invention relates to human actin-binding regulatory proteins, nucleic acids encoding these proteins, epitopes of these proteins and antibodies specific for these epitopes. The invention also relates to screening methods for the identification of potential drug candidate molecules and the use of such molecules in cancer therapy and the treatment of other disorders of cell motility, cell proliferation, wound healing, growth and division.

### BACKGROUND

The conversion of epithelial cells from sessile, non-dividing cells in monolayers to motile, proliferating cells of invasive carcinomas must be tightly coupled to highly regulated rearrangements of the actin cytoskeleton. Whether epithelial transformation requires expression of new cytoskeleton components or merely rearrangement of the existing epithelial cytoskeleton remains open to question. In malignant carcinoma tumors, invasion of transformed epithelial cells into the underlying connective tissue occurs by cell migration.

Metastasis of carcinoma tumors also involves cell migration from the primary tumor site into blood vessels by diapedesis through the vessel endothelium. Migration of metastatic tumor cells was clearly described by Waldeyer in 1872 as amoeboid movement (Weiss, L. 1985. Principles of Metastasis. Academic Press, Inc., Orlando, Fla., p26) a form of cell motility that requires coordinated mobilization and remodeling of the actin cytoskeleton by actin-binding proteins. An initial step in cortical actin cytoskeleton rearrangement includes site-specific actin polymerization onto actin filament ends that have been generated by severing or uncapping of existing filaments. Two families of actin filament fragmenting/capping proteins are presently recognized, the severin/fragmin/gelsolin family containing shared 125 amino acid repeat domains and the actin depolymerization factor family of ADF, depactin, destrin, and actophorin.

Severin from Dictyostelium amoebae and fragmin in Physarum slime molds are the earliest phylogenetic examples of actin filament fragmenting proteins. The parallel actin severing protein in mammalian cells is gelsolin, an 80 kDa protein reported to be derived from duplication of the ancestral severin gene. A cytoplasmic gelsolin is expressed in epithelial cells, fibroblasts and leucocytes, and secreted plasma gelsolin is present in blood. In gelsolin, it is the conservation of severin amino acid sequences that accounts for the actin filament severing activity.

Paradoxically, despite the heightened migratory behavior of invasive tumor cells, gelsolin is extensively down-regulated during transformation of mammary epithelium and fibroblasts. Mammalian actin-binding regulatory proteins that are present and not down-regulated in tumor cells and neoplastic transformed cells were not known prior to the present invention.

### SUMMARY OF THE INVENTION

The present invention provides isolated actin-binding regulatory proteins which are expressed motile, proliferating and invasive cells, in tumors and in neoplastic transformed cells and in cells at the site of a wound and are up-regulated in tumor cells. In a particular embodiment the actin-binding regulatory protein is H-severin. In a second embodiment the protein is H-30.

In yet another embodiment the invention provides an actin-binding regulatory protein expressed in motile, proliferating and invasive cells, and in cells at a wound site or at the site of a healing wound, which is capable of severing F-actin filaments.

In another embodiment the invention provides nucleic acid molecules encoding the actin-binding regulatory molecules described herein above. Also provided are nucleic acid vectors and host cells comprising the nucleic acid molecules which encode the actin-binding regulatory molecules.

The invention also provides antibodies which specifically bind an epitope of an actin-binding regulatory protein. In particular embodiments these antibodies are monoclonal and polyclonal antibodies which specifically bind H-severin and H-30.

The invention further provides a method of determining the proliferative status or stage of carcinogenesis of a human cell, which may be a pre-cancerous condition. The steps involved in the test are as follows: providing a human cell sample, assessing the level of expression or activity of a human actin-binding regulatory protein in at least one cell of the cell sample, wherein a high level of expression or activity of the human actin-binding regulatory protein correlates with cellular proliferation, motility, neoplasia or wound healing, thereby determining the proliferative status or stage of carcinogenesis of the cell. In one embodiment the level of actin-binding regulatory protein, may be assessed by northern blot or western blot, or by cytoimmuno-histochemistry.

In yet a further embodiment the invention provides a method of identifying a test compound as a modulator of expression or activity of an actin-regulatory protein, comprising: providing a first cell expressing the actin-regulatory protein and having actin-regulatory protein activity; contacting the first cell with the test compound; assessing the level of expression or activity of the actin-regulatory protein in the first cell; providing a second cell identical to the first cell expressing actin-regulatory protein and having actin-regulatory protein activity, which has not been contacted with the test compound; assessing the expression or activity of the actin-regulatory protein in the second cell; and comparing the expression or the activity of M-severin in the first and second cell, thereby determining whether the test compound is a modulator of actin-regulatory protein expression or activity.

In yet another embodiment the invention provides a method of treating a human cell in a stage of carcinogenensis comprising: administering an effective amount of a compound which modulates H-severin expression or activity such that carcinogenesis is modulated. Among these compounds are molecules that modulate the actin-binding or actin-severing activity of the H-severin. Other compounds of this embodiment may function by modulating the transcription of the H-severin.

Further in yet another embodiment the invention provides a method of inhibiting proliferation of a human cell in a stage of carcinogenensis comprising administering to the cell an effective amount of a compound which inhibits human actin-binding regulatory protein expression or activity such that the carcinogenesis is inhibited.

### BRIEF DESCRIPTION OF THE FIGURES

FIG. 1. Predicted amino acid sequence of cDNA clone 10c-1. The predicted amino acid sequence of the protein encoded by clone 10c-1 was determined from the open reading frame (ORF) of the partial cDNA sequence which encodes 136 amino acids.

FIG. 2. M-severin mRNA Expression Increases with Metastatic Potential of Epithelial Cells. Comparative expression patterns of M-Severin mRNA in transformed cells of increasing metastatic potential. Equivalent amounts (50 μg) of total RNA prepared from each cell type was probed with a 0.65 kb cDNA of M-severin cloned from a P-19 carcinoembryonic cell library. Blots were exposed for 36 h (1×exposure) followed by a 14 day exposure (10×) in a Molecular Dynamics PhosphorImager. A 1.9 kb signal was common to all cell types. Signal strength increased with metastatic potential of the cell line. Highly metastatic P19 cells (b) expressed approximately 7-fold more M-severin mRNA than weakly metastatic LL/2 cells (c) and 70-fold more M-severin than immortalized MDCK cells (d).

FIG. 3. Localization of M-severin in LL/2 cells. Cultured LL/2 cells labeled for M-severin (a, c) or F-actin (b) were examined by confocal microscopy. a.) M-severin cytoimmunofluoresence in a dividing and motile LL/2 cell. b.) F-actin rhodamine phalloidin staining in a dividing LL/2 cell. M-severin and F-actin colocalized to the leading cell edge and cleavage furrow. c.) The dividing cell pair shown in panel (a) in vertical section through the cell midline confirms the highest concentrations of M-severin at the leading cell edge and cleavage furrow.

FIG. 4. Expression of H-severin in human colon adenocarcinomas. Resected adenocarcinoma tumors from 2 patients were examined for the presence of H-severin in tranformed epithelium and in motile cells of the colonic connective tissue. a, b) Colon serial sections through well-differentiated adenocarcinoma in colonic villi (CV) and underlying connective tissue (CT) containing moderately differentiated adenocarcinoma tumor (AT). a) Control staining with secondary HRP-conjugated antibody and hematoxylin shows cell nuclei (blue) only. b) H-severin staining with hematoxylin counterstain shows H-severin expression in the basal aspect of epithelial cells of well-differentiated adenocarcinoma (CV), throughout cells of moderately differentiated adenocarcinoma (AT), and in fibroblasts of the connective tissue (CT). c, d) Low (c, 100×) and high (d, 600×) magnification of normal colon epithelium at the surgical margin of a resected tumor. Normal epithelial cells carry hematoxylin stained nuclei, but do not express H-severin (d, arrows). H-severin staining is apparent in fibroblasts (d, *) of the lamina propria subjacent to the basement membrane of colonic epithelial cells. e,f) Low power (e, 100×) and high power (f, 600×) magnification of moderately differentiated adenocarcinoma containing H-severin. g, h) Low power (g, 10×) and high power (h, 600×) magnification of undifferentiated adenocarcinoma (g, arrow) showing heavy expression of H-severin. Comparison of normal epithelium with moderately differentiated adenocarcinoma and undifferentiated carcinoma (d, f and h, respectively) indicates enhanced expression of H-severin in advancing stages of tumor progression.

FIG. 5. H-30 in Human Colon Adenocarcinomas. Cytohistochemical HRP staining with Anti-M30 (brown) and nuclear counterstaining with hematoxylin (blue) of a resected colon adenocarcinoma tumor from a single patient. a,c,e,g,i) 100×magnification. b, d, f, h, j) 600×magnification. a, b) Normal colon mucosa from surgical margin of the resected colon showing cross-sections through epithelial villi and underlying lamina propria (* in b). Epithelial mucosal cells show basal nuclei and the absence of M-30 Protein. c,d) Well-differentiated dysplasia of epithelial mucosa with cuboidal epithelial cells expressing M-30. Slight M-30 staining of fibroblasts in underlying lamina propria connective tissue is also evident (*). e, f, g, h) Moderately differentiated adenocarcinoma. Epithelium contains dividing cells and abundant expression of M-30 Protein. i, j) Undifferentiated adenocarcinoma with heavy M-30 Protein staining, loss of epithelial monolayer and proliferating undifferentiated epithelial cells. M-30 Protein continues to be expressed during progressive stages of colon adenocarcinoma tumorigenesis.

FIG. 6. Colocalization of M30 Protein with Tyrosine Phosphate and F-Actin and in the Cleavage Furrow of Mitotic LLC Cells. Dual staining for M30 and either F-actin or Phosphotyrosine were used to create horizontal colocalized confocal images through the floor of a dividing cell. M30 (green) and either tyrosine phosphate (red) (a), or actin (red) (c), shows colocalization in yellow. Similar overlays were created for vertical cross-sections through the cell's cleavage furrow for M30 with tyrosine phosphate (b) and actin (d). Scale bar equals 4 μm.

### DETAILED DESCRIPTION OF THE INVENTION

The present invention is based on the surprising discovery that in mammals a previously unknown class of actin-binding regulatory proteins function to achieve actin filament rearrangements necessary for active cell migration in invasive or metastatic carcinomas. The class includes severin and M-30. The previously-studied 80Kd actin-binding regulatory protein, gelsolin was heretofore the prime candidate as the mediator of actin rearrangements. However, gelsolin is down-regulated in mammalian tumor cells, whereas severin and M-30 activities are herein shown to be increased in invasive human adenocarcinoma as revealed by immunocytochemical staining of severin with antibodies directed against Dictyostelium severin (Anti-DdSev) and the Dictyostelium 30 Kd actin-binding protein. The present invention discloses the detection by cytoimmunohistochemical staining of H-severin and of H30 induced in an invasive human colon adenocarcinoma. Since normal colon epithelium from the same patient expressed neither H-severin nor H-30, these serve as sensitive markers for detection and staging of tumors, such as epithelial tumors.

The present invention provides a new class of actin-binding regulatory proteins. Actin-binding regulatory proteins are proteins that bind to actin, and that regulate the bundling, crosslinking, cleavage or dissociation of the actin network in a cell. Changes in bundling and cross-linking of actin within a cell are responsible for changes in cell shape and cell movement in such diverse physiological phenomena as: cell locomotion (as in formation of pseudopodia and lamellipodia), cell proliferation, growth, division, and chemotaxis and tissue invasion (for example in invasion by a metastatic tumor) and also in assembly, formation, modification or destruction of the cellular actin network.

The human actin-binding regulatory proteins are expressed in motile, proliferating and invasive cells, and in cells at the site or a wound. Further, these proteins are up-regulated in tumor cells. For the purpose of this specification, a tumor cell is a transformed neoplastic cell. The tumor cells may be malignant or non-malignant. Tumor cells may be from all classes of tumors, such as carcinomas, sarcomas, gliomas, melanomas, osteosarcomas, hepatomas etc.

The up-regulation of actin-binding regulatory proteins in tumor cells means that the level of expression or activity of these proteins is increased in tumor cells. Increased expression of these proteins from the genes encoding them means that the level of protein produced from the genes encoding the actin-binding regulatory proteins is increased in these cells. This increased expression may be achieved by increased gene transcription to produce mRNA and/or increased translation to produce the primary protein translation product, or by increased stability of the protein (such as for example, by inhibition of degradation by specific proteases).

Expression of the actin-binding regualtory protein may require processing of the primary protein translation product. The primary protein translation product may be matured by cleavage and/or any one or more of a number of post-translational modifications. Cleavage of the primary protein product may be to remove signal sequences and/or pro-protein sequences to produce mature protein chains. The mature protein chains may be post-transcriptionally modified in any of a number of ways well known to those of skill in the art, including but not limited to phosphorylation, methylation, acylation and prenylation as well as cross-linking of sulfhydryls and covalent cross-linking to other proteins or peptides.

Increased activity of actin-binding regulatory proteins in tumor cells refers to an increase in any of the activities of these proteins in tumor cells. Such activities include, for example, actin-binding activity, actin-bundling activity, actin-severing activity or other actin modifying activity mediated by the actin-binding regulatory protein.

The expression or activity of these actin-binding regulatory proteins is increased in tumor cells. Previously known actin-binding proteins, such as the 80 Kd actin filament-cleaving protein, gelsolin are not expressed at higher levels in tumor cells than in normal cells, nor do tumor cells exhibit higher gelsolin activity. Therefore, gelsolin is not an “actin-binding regulatory molecule” according to the definition of the present invention.

In one embodiment the actin-binding regulatory proteins are isolated. In the context of the present invention, a protein is isolated if the protein is substantially free of other cellular proteins. Preferably, an isolated protein is purified to single band purity as judged on a coomassie blue stained SDS-acrylamide gel. Even more preferably, the isolated protein is purified to single band purity as judged on a silver stained SDS-acrylamide gel.

In a particular embodiment, the actin-binding regulatory protein is H-severin. H-severin is a human protein that cross-reacts with antibodies directed against the Dictyostelium form (DdSev) and the mouse form of mammalian severin (M-severin). H-severin has an apparent MW of approximately 40 Kd as assessed by SDS-polyacrylamide gel electrophoresis. H-severin is an actin-binding protein with actin-cleaving (severing) properties. H-severin regulates the cellular actin network in vivo by selectively binding, bundling, and cleaving (severing) the actin molecules in a cell. The H-severin protein is particularly highly expressed in advanced stage tumor cells, somewhat less well expressed in adenocarcinoma cells and even less well expressed in well differentiated pre-adenocarcinoma cells.

In another particular embodiment, the actin-binding regulatory protein is H-30. H-30 protein is a human actin-bundling protein that cross reacts with both anti-Dictyostelium M-30 antibodies and anti-mouse M-30 antibodies and exhibits an apparent MW of approximately 34 Kd to 35 Kd as assessed on SDS-polyacrylamide gels. H-30 binds to actin molecules within the cell and regulates the actin network of the cell by mechanisms involving activation of the H-30 protein either by or coincident with the phosphorylation of certain tyrosine residues of H-30. Modulation or inhibition of H-30 according to the methods of the present invention may be achieved by modulation or inhibition of the expression or of the actin-binding activity of H-30, or of the phosphorylation of tyrosine residues of H-30

The actin-binding regulatory proteins described herein include H-severin, H-30 and all of the naturally occurring alleles and polymorphs of the human actin-binding regulatory proteins expressed in motile, proliferating and invasive human cells, and in human cells at a wound site. Also included are fragments of H-severin and H-30 that retain actin-binding, actin-severing or actin regulatory function or any combination of these properties.

It should be understood that throughout the specification the terms M-severin and M-30 refer to the mammalian severin protein and the mammalian 30 Kd protein (which cross react with antibodies against the Dictyostelium 30 Kd protein), of which the human H-severin and H-30 are species. Further, the terms H-severin and H-30 refer to both native and recombinant forms of the proteins whether isolated from human cells or from recombinant hosts, which may be eukaryotic or prokaryotic hosts carrying the recombinant nucleic acid sequence encoding H-severin or H-30.

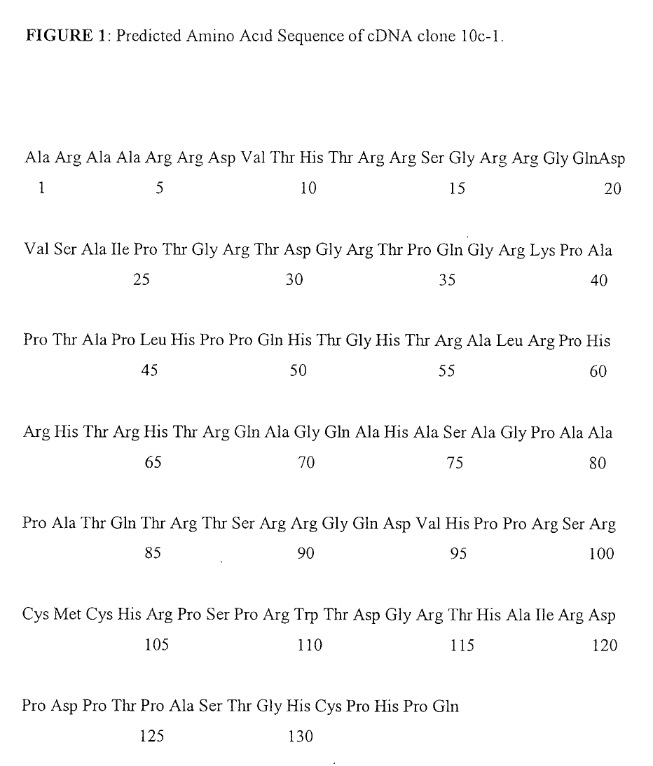

A further distinguishing characteristic of H-severin is that it has a sequence that is homologous to the murine form, a partial sequence of which is shown in FIG. 1 (SEQ ID NO: 1). A protein is homologous to a second protein if the two proteins are at least approximately 75%, preferably at least approximately 80%, more preferably at least approximately 85%, most preferably at least about 90%, and optimally at least approximately 95% to each other.

In order to compare a first amino acid or nucleic acid sequence to a second amino acid or nucleic acid sequence for the purpose of determining homology, the sequences are aligned so as to maximize the number of identical amino acid residues or nucleotides. The sequences of highly homologous proteins and nucleic acid molecules can usually be aligned by visual inspection. If visual inspection is insufficient, the nucleic acid molecules may be aligned in accordance with the methods described by George, D. G. et al., in Macromolecular Sequencing and Synthesis, Selected Methods and Applications, pages 127-149, Alan R. Liss, Inc. (1988), such as formula 4 at page 137 using a match score of 1, a mismatch score of 0, and a gap penalty of −1.

Human actin-binding regulatory proteins may be isolated from from any human cell. For example, the actin-binding regulatory molecules may be isolated from human cells from any tissue including skeletal muscle, heart muscle, other smooth or striated muscle tissues; hematopoietic cells or cells from tissues or from organs such as lung or kidney may also be used. Human lung cells are especially preferred. Alternatively, the actin-binding regulatory molecules may be isolated from any readily available human cell source such as human hematopoietic cells. The cells expressing human actin-binding regulatory proteins may be normal cells, including motile and proliferating cells, or they may be tumor cells. The H-severin protein is particularly highly expressed in advanced stage tumor cells.

Further, nucleic acid molecules encoding actin-binding regulatory molecules are provided by the present invention. The nucleic acid molecule may be DNA or RNA and encodes the amino acid sequence of the primary protein product, the mature protein product or active fragment of H-severin or H-30. The nucleic acid molecule encoding the actin-binding regulatory protein or active fragment may be isolated or may be carried on a recombinant vector.

The recombinant vector carrying the nucleic acid encoding the actin-binding regulatory protein or active fragment may be transformed into a recombinant host cells. Further the recombinant host may express the actin-binding regulatory protein or active fragment from the recombinant vector. The recombinant host cell may be prokaryotic or eukaryotic. Some examples of each include bacterial cells such as E.coli or Salmonella cells; eukaryotes such as the yeast Saccharomyces or the filamentous fungus, Aspergillus; insect cells such as Spodoptera frugiperda Sf9 cells or mammalian cells, such as human, murine, rat or rabbit cells from primary isolates or cell lines in culture. In particular embodiments the nucleic acid molecules encoding actin-binding regulatory molecules encode H-severin or H-30.

The actin-binding regulatory proteins of the invention may be used for various purposes. For example, the proteins may be used to raise antibodies. The antibodies may be polyclonal, monoclonal, single chain antibodies or antibody fragments that retain the ability to bind antigen. Among the monoclonal and polyclonal antibodies and single chain antibodies and antibody fragments that retain the ability to bind antigen of the present invention are those which specifically or selectively bind H-severin or H-30. These antibodies are useful, for example for isolating and purifying the proteins, for identifying cells and biological samples that contain the proteins, and for diagnosis and staging of metastatic disease.

As used herein, the term “specifically binds” in referring to eptope binding means binds only to the epitope in question.

The H-severin is cross reactive with antibodies raised against Dictyostelium severin and also with antibodies raised against mouse M-severin and exhibits actin-binding and actin-severing activities.

Specifically, the isolation and uses of H-severin and the H-30 are presented. These proteins may be used in the methods of the present invention to raise antibodies for diagnosis and staging of metastatic disease, for assays and screens for compounds which modulate actin-binding, actin-severing or other actin regulatory functions. Compounds (modulators, including inhibitors) identified by such assays and screens may be used in management and treatment of disorders of cell proliferation, growth and metastasis by inhibiting H-severin function or H-30 function. Alternatively, wound healing may be accelerated by enhancing the function of H-severin or of H-30.

As shown in the examples below, invasive human colon adenocarcinoma tumors contain abundant levels of H-severin, and M-30 while normal colon epithelium from the same patient do not express the protein. Thus, lysates of highly motile and transformed epithelial LL/2 cells together with their resultant Lewis lung carcinoma tumors were analyzed for the presence of severin, the ancestral actin filament fragmentation protein prominent in Dictyostelium amoebae. The results indicate that both LL/2 cells and their derived tumors contain a mammalian form of severin. Moreover, while gelsolin is dominantly expressed in normal lung epithelium, H-severin appears to become expressed during transformation to replace gelsolin in LL/2 cells and tumors. Furthermore, M-severin expression appears to be a general feature of motile and/or transformed epithelial cells, but not of non-motile cells of muscle, liver or normal epithelium. It is this specificity for motile cells that makes M-severin/H-severin useful for marking invasive tumors, such as, for example, invasive carcinoma tumors.

Accordingly, in one embodiment, the invention is directed to a method of determining the proliferative status or stage of carcinogenesis of a cell. In the first step, a cell sample is provided.

The cell sample may be from any cell source, for example from a cell culture, primary cell isolate or biopsy. For example the cells may be normal cells, pre-cancerous cells or cells from a tumor.

Normal cells are cells that show no significant signs of growth disorder, neoplasia or cellular changes normally associated with carcinogenesis.

Pre-cancerous cells are cells that are committed to developing into tumor cells. Some examples of pre-cancerous cells include cells from colo-rectal polyps, cells from cells from prostate tissue having a high PSA (Prostate Specific Antigen) level or cells from APC (Adenomatous Polyposis Coli) positive tissue.

As mentioned above, for the purpose of this specification, a tumor cell is a transformed neoplastic cell. The tumor cells may be malignant or non-malignant and may be from all classes of tumors, such as carcinomas, sarcomas, gliomas etc.

In the second step of the method for determining the prolierative status or stage of carcinogenesis of a cell the level of expression or activity of H-severin, H-30 or tyrosine phosphorylated H-30 is assessed. Expression of H-severin, H-30 or tyrosine phosphorylated H-30 may be assessed by techniques known in the art.

For example, transcription assays employing reporter genes coupled to the promoter of the monitored gene may be used. Any gene which has a detectable product may be used as a reporter gene in the methods of the present invention. Preferred reporter genes are those for which routine assays are readily available.Some useful reporter genes include, for example alkaline phosphatase (AP), luciferase (luci), chloramphenicol acetyl transferase (CAT), β-galactosidase (lacZ), and β-lactamase (bla).

The level of expression of H-severin, H-30, or tyrosine-phosphorylated H-30 may also be assessed by standard blot techniques or by cytoimmunohistochemistry. Some standard blot techniques include, for example, northern blot or western blot techniques.

The level of activity of H-severin, H-30, or tyrosine-phosphorylated H-30 may also be assessed by standard techniques. The activities of these proteins have been discussed above.

The proliferative status or stage of carcinogenesis of the cell is correlated with the level of expression and/or activity of H-severin, H-30, or tyrosine-phosphorylated H-30. A high level of any of these proteins correlates with cellular proliferation, motility, neoplasia or wound.

In another embodiment, the actin-binding regulatory proteins are useful in assays and screens for compounds which modulate actin-binding, actin-severing or other actin regulatory functions. Compounds (modulators) identified by such assays and screens may be used in management and treatment of disorders and diseases such as cancer, characterized by excessive cell proliferation, motility, and metastasis by inhibiting H-severin or H-30 function. Alternatively, wound healing may be accelerated by enhancing the function of H-severin or of H-30.

In the first step of the assays and screens, a first cell expressing H-severin, H30, or tyrosine-phosphorylated H-30 is provided. The cell may be any cell that expresses H-severin, H-30, or tyrosine-phosphorylated H-30. The cell may be a naturally occurring cell, or a recombinant cell.

Naturally occurring cells are preferably mammalian tumor cells, more preferably murine or human tumor cells. Some examples of suitable tumor cells are provided below. The cells may be primary cells or may constitute a cell line.

Recombinant cells useful in the method include any cell that is transfected with the gene for H-severin, H-30, or tyrosine-phosphorylated H-30. The recombinant cells are preferably mammalian cells, more preferably rodent, such as mouse and primate, such as human cells. Some examples of suitable mammalian cell include, for example, CHO cells and COS cells, HEK293 cells and A549 cells.

In the second step, the first cell is contacted with a test compound. Contacting the cell may be achieved by any method known in the art. For example, the cell may be contacted with the test compound by adding an effective amount of the compound directly to a culture medium containing the cells. If the cell is present in an intact animal, the contacting may be achieved by administering an effective amount of the compound in a pharmaceutically acceptable carrier. The test compound may be administered by any means, such as for example, intravenously (i.v.), interperitoneally (i.p.), or (p.o.).

In the third step, the level of expression or activity of the H-severin, H30, or tyrosine-phosphorylated H-30 in the first cell is assessed by methods described above.

In the fourth step, a second cell is provided. The second cell is identical to the first cell, except that the second cell acts as a control cell, and is not contacted with the test compound. In the fifth step, the level of expression or activity of the H-severin, H-30, or tyrosine-phosphorylated H-30 in the second cell is assessed by methods described above.

In the final step, the level of expression or activity of H-severin, H-30, or tyrosine-phosphorylated H-30 in the first and second cell is compared. If there is a significant difference between the level of expression or activity of H-severin, H-30, or tyrosine-phosphorylated H-30 in the first and second cell, the test compound is identified as a modulator of H-severin, H-30, or tyrosine-phosphorylated H-30. A modulator of H-severin, H-30, or tyrosine-phosphorylated H-30 can be either a stimulator or an inhibitor of the activity of the protein.

The modulators of actin-binding regulatory molecules identified by the methods of the present invention may be small molecules or biological molecules. Such biological molecules include all lipids and polymers of monosaccharides, amino acids and nucleotides having a molecular weight up to 300 or even 450 daltons. Thus, biological molecules include, for example, fragments of oligosaccharides and polysaccharides; oligopeptides, polypeptides, peptides, and proteins; and oligonucleotides and polynucleotides. Oligonucleotides and polynucleotides include, for example, DNA and RNA.

Biological molecules further include derivatives of any of the molecules described above. For example, derivatives of biological molecules include lipid and glycosylation derivatives of oligopeptides, polypeptides, peptides and proteins. Derivatives of biological molecules further include lipid and glycosylated derivatives of oligosaccharides and polysaccharides, e.g. lipopolysaccharides.

Any molecule that is not a biological molecule is considered in this specification to be a small molecule. Accordingly, small molecules include organic compounds, organometallic compounds, salts of organic and organometallic compounds, saccharides amino acids, and nucleotides. Small molecules further include molecules that would otherwise be considered biological molecules, except their molecular weight is not greater than 450. Thus, small molecules may be lipids, oligosaccharides, oligopeptides, and oligonucleotides, and their derivatives, having a molecular weight of 450 or less.

It is emphasized that small molecules can have any molecular weight. They are merely called small molecules because they typically have molecular weights less than 450 daltons.

In the methods described above, expression of H-severin, H-30 and other actin binding proteins may also be monitored by transcription assays employing reporter genes coupled to the promoter of the monitored gene. Reporter genes useful for this embodiment of the methods of the present invention include alkaline phosphatase (AP), luciferase (luci), chloramphenicol acetyl transferase (CAT), β-galactosidase (lacZ), and β-lactamase (bla). This list is intended only as a guide and should not be construed as limiting in any way. Any gene which has a detectable product may be used as a reporter gene in the methods of the present invention; especially preferred are those such as AP, luci, CAT, lacZ, and bla for which routine assays are readily available.

Several methods well known in the art may be used to detect, monitor or measure the effect of compounds active on the expression or activity of human actin-binding regulatory molecules on cell growth or proliferation. Modulation, such as inhibition of proliferation may be assessed qualitatively as a detectable change in growth or proliferation, or quantitatively wherein the detectable change is the difference between a measured proliferation parameter (such as incorporation of ³H-thymidine from ³H-TTP into chromosomal DNA) the test cell contacted with the test compound and in an identical control untreated cell. Inhibition of proliferation may be scored as detectable in the qualitative assay, or as a 10%, or preferably 50% or 80%, or most preferably 100% inhibition of proliferation in the treated cell as assessed by a quantitative assay.

The screening methods contemplated in the invention include for example, cell-free systems in which the components may be obtained from the tissues of an organism, primary cells, cultured cell lines or from recombinant cells. Prokaryotic organisms including for example: Escherichia coli and Salmonella typhimurium may be used as recombinant hosts for the production of any or all of the following: Actin-binding regulatory proteins, H-severin or H-30 components specified in the invention. Eukaryotic organisms including yeast (e.g. Saccharomyces cerevisiae), the filamentous fungus Aspergillus, and insect cells (e.g. sf9 cells of Spodoptera frugiperda), or mammalian cell lines as disclosed herein may also be useful for production of the components used in the methods of this invention. These components may be used in cell-free systems derived from these eukaryotic organisms. Alternatively, these methods, particularly the screening methods, may be carried out using cells in culture or directly in the intact organism.

The invention also provides a method of treatment of a human cell, having a proliferating tumor or other growth regulation disorder which comprises: administering to the human cell an effective amount of a compound capable inhibiting the expression or activity of H-severin, or of H-30 such that the proliferation of the tumor is inhibited or the growth regulation disorder is ameliorated. Administration of the compound as described above, may be for example, intravenous, intraperitoneal or oral, such as with solid food or liquids, syrups etc. or in mixtures comprising approved (GRAS: generally regarded as safe- for use in foods) carriers.

An effective amount of a compound is that amount which upon contacting the cell leads to a detectable change in proliferation or tumor growth and most preferably leads to total suppression of proliferation while causing minimal or no unwanted side-effects in the cell or in the whole animal. An effective amount of compound per weight of cells or body weight may be between 1 and 100 ng/kg, but is preferably between 1 and 100 ug/kg, or between 1 and 100 mg/k, but may also be 1 gm/kg or even 10 gm/kg body weight.

The cell may be in vivo in an intact animal or human, or ex-vivo in an explant cell sample, tissue or organ. The ex-vivo treated cells may then be re-introduced into the intact animal or human.

### EXAMPLES

### General Methods

Preparation of Protein

The protein and fragments of the present invention may be prepared by methods known in the art. Such methods include isolating the protein directly from cells, isolating or synthesizing DNA encoding the protein and using the DNA to produce recombinant protein, and synthesizing the protein chemically from individual amino acids.

A. Isolation of Protein from Solution

Proteins are isolated from the solubilized fraction by standard methods. Some suitable methods include precipitation and liquid/chromatographic protocols such as ion exchange, hydrophobic interaction and gel filtration See, for example, Guide to Protein Purification, Deutscher, M. P. (Ed.)_Methods Enzymol., 182, Academic Press, Inc., New York (1990) and Scopes, R. K. and Cantor, C. R. (Eds.), Protein Purification (3d), Springer-Verlag, New York (1994).

B. Isolation of Protein from Gels

Alternatively, purified material is obtained by separating the protein on preparative SDS-PAGE gels, slicing out the band of interest and electroeluting the protein from the polyacrylamide matrix by methods known in the art. The detergent SDS may be removed from the protein by known methods, such as by dialysis or the use of a suitable column, such as the Extracti-Gel column from Pierce Chemical Company.

C. Chemical Synthesis of Protein

The proteins of the invention and DNA encoding the proteins may also be chemically synthesized by methods known in the art. Suitable methods for synthesizing the protein are described by Stuart and Young in “Solid Phase Peptide Synthesis,” Second Edition, Pierce Chemical Company (1984), Solid Phase Peptide Synthesis, Methods Enzymol., 289, Academic Press, Inc, New York (1997). Suitable methods for synthesizing DNA are described by Caruthers in Science 230:281-285 (1985) and DNA Structure, Part A: Synthesis and Physical Analysis of DNA, Lilley, D. M. J. and Dahlberg, J. E. (Eds.), Methods Enzymol., 211, Academic Press, Inc., New York (1992).

Recombinant Protein

The protein may also be prepared by providing DNA that encodes the protein; amplifying or cloning the DNA in a suitable host; expressing the DNA in a suitable host; and harvesting the protein and in certain embodiments. purifying the protein.

A. Providing DNA

1. Chemical Synthesis from Nucleotides

The DNA may be synthesized chemically from the four nucleotides (A, T. G and C) in whole or in part by methods known in the art. Such methods include those described by Caruthers in Science 230:281-285 (1985) and DNA Structure, Part A: Synthesis and Physical Analysis of DNA, Lilley, D. M. J. and Dahlberg, J. E. (Eds.), Methods Enzymol., 211, Academic Press, Inc., New York (1992).

Alternatively, the nucleic acid molecules of the invention may be isolated from the available cDNA libraries and screened with selected probes designed to identify the gene of interest. See Sambrook, J. et al. (eds), Molecular Cloning, Second Edition, Cold Spring Harbor Laboratory Press, Cold Spring Harbor, N.Y. (1989) and Ausubel, F. M. et al. (eds), Current Protocols in Molecular Biology, John Wiley & Sons, New York (1999).

DNA may also be synthesized by preparing overlapping double-stranded oligonucleotides, filling in any gaps with polymerase I, and ligating the ends together with DNA ligase. The DNA may be cloned in a suitable host cell and expressed in the same cell or isolated and transformed in a host cell more suitable for expression. The DNA and protein may be recovered from the host cell. See, generally, Sambrook, J. et al. (Eds.), Molecular Cloning, Second Edition, Cold Spring Harbor Laboratory Press, Cold Spring Harbor, N.Y. (1989) and _Ausubel, F. M. et al. (Eds.), Current Protocols in Molecular Biology, John Wiley & Sons, Inc., New York (1999).

B. Expressing DNA

The DNA encoding the protein of the invention may be replicated and used to express recombinant protein following insertion into a wide variety of host cells in a wide variety of cloning and expression vectors. The host may be prokaryotic or eukaryotic. The DNA may be obtained from natural sources and, optionally, modified. The genes may also be synthesized in whole or in part.

Cloning vectors may comprise segments of chromosomal, non-chromosomal and synthetic DNA sequences. Some suitable prokaryotic cloning vectors include plasmids from E. coli, such as colE1, pCR1, pBR322, pMB9, pUC, pKSM, and RP4. Prokaryotic vectors also include derivatives of phage DNA such as lambda and M13 or fd, and other filamentous single-stranded DNA phages.

Vectors for expressing proteins in bacteria, especially E.coli, are also known. Such vectors include the pK233 (or any of the tac family of plasmids), T7, pBluescript II, bacteriophage lambda ZAP, and lambda P_{L} (Wu, R. (Ed.), Recombinant DNA Methodology II, Methods Enzymol., Academic Press, Inc., New York, (1995)). Examples of vectors that express fusion proteins are PATH vectors described by Dieckmann and Tzagoloff in J. Biol. Chem. 260, 1513-1520 (1985). These vectors contain DNA sequences that encode anthranilate synthetase (TrpE) followed by a polylinker at the carboxy terminus. [see addition from SKE-1-P] Other expression vector systems are based on beta-galactosidase (pEX); maltose binding protein (pMAL); glutathione S-transferase (PGST or PGEX)—see Smith, D. B. Methods Mol. Cell Biol. 4:220-229 (1993); Smith, D. B. and Johnson, K. S., Gene 67:31-40 (1988); and Peptide Res. 3:167 (1990), and TRX (thioredoxin) fusion protein (TRXFUS)—see LaVallie, R. et al., Bio/Technology 11:187-193 (1993).

Vectors useful for cloning and expression in yeast are available. Suitable examples are 2 μm circle plasmid, Ycp50, Yep24, Yrp7, Yip5, and pYAC3 (Ausubel, F. M. et al. (Eds.), Current Protocols in Molecular Biology, John Wiley & Sons, Inc., New York, (1999)).

Suitable cloning/expression vectors for use in mammalian cells are also known. Such vectors include well-known derivatives of SV-40, adenovirus, cytomegalovirus (CMV) retrovirus-derived DNA sequences. Any such vectors, when coupled with vectors derived from a combination of plasmids and phage DNA, i.e. shuttle vectors, allow for the isolation and identification of protein coding sequences in prokaryotes.

Further eukaryotic expression vectors are known in the art (e.g., P. J. Southern and P. Berg, J. Mol. Appl. Genet. 1:327-341 (1982); S. Subramani et al, Mol. Cell. Biol. 1:854-864 (1981); R. J. Kaufmann and P. A. Sharp, “Amplification And Expression Of Sequences Cotransfected with A Modular Dihydrofolate Reductase Complementary DNA Gene,” J. Mol. Biol. 159:601-621 (1982); R. J. Kaufinann and P. A. Sharp, Mol. Cell. Biol. 159:601-664 (1982); S. I. Scahill et al, “Expression And Characterization of The Product of a Human Immune Interferon DNA Gene In Chinese Hamster Ovary Cells,” Proc. Natl. Acad. Sci. USA 80:4654-4659 (1983); G. Urlaub and L. A. Chasin, Proc. Natl. Acad. Sci. USA 77:4216-4220 (1980).

The expression vectors useful in the present invention contain at least one expression control sequence that is operatively linked to the DNA sequence or fragment to be expressed. The control sequence is inserted in the vector in order to control and to regulate the expression of the cloned DNA sequence. Examples of useful expression control sequences are the lac system, the tm system, the tac system, the trc system, the tet system, major operator and promoter regions of phage lambda, the control region of fd coat protein, the glycolytic promoters of yeast, e.g., the promoter for 3-phosphoglycerate kinase, the promoters of yeast acid phosphatase, e.g., Pho5, the promoters of the yeast alpha-mating factors, and promoters derived from polyoma, adenovirus, retrovirus, and simian virus, e.g., the early and late promoters or SV40, and other sequences known to control the expression of genes of prokaryotic or eukaryotic cells and their viruses or combinations thereof.

Useful expression hosts include well-known prokaryotic and eukaryotic cells. Some suitable prokaryotic hosts include, for example, E. coli, such as SG-936, HB 101, W3110, X1776, X2282, DH1, DH5αF′, and MRC1, Pseudomonas, Bacillus, such as Bacillus subtilis, and Streptomyces. Suitable eukaryotic cells include yeasts and other fungi, insect, animal cells, such as COS cells and CHO cells, human cells and plant cells in tissue culture.

C. Fusion Proteins

The proteins of the invention may be expressed in the form of a fusion protein with an appropriate fusion partner. The fusion partner preferably facilitates purification and identification. Increased yields may be achieved when the fusion partner is expressed naturally in the host cell. Some useful fusion partners include beta-galactosidase (Gray, et al., Proc. Natl. Acad. Sci. USA 79:6598 (1982)); trpE (Itakura et al., Science 198:1056 (1977)); protein A (Uhlen et al., Gene 23:369 (1983)); glutathione S-transferase (Smith, D. B., Methods Mol. Cell Biol. 4:220-229 (1993); Smith, D. B. and Johnson, K. S., Gene 67:31-40 (1988); Johnson, Nature 338:585 (1989)); Van Etten et al., Cell 58:669 (1989)); and maltose-binding protein (Guan et al., Gene 67:21-30 (1987); Maina et al., Gene 74:36-373 (1988), in Ausubel, F. M. et al. (Eds.) Current Protocols in Molecular Biology, John Wiley & Sons, Inc., New York (1999)).

Such fusion proteins may be purified by affinity chromatography using reagents that bind to the fusion partner. The reagent may be a specific ligand of the fusion partner or an antibody, preferably a monoclonal antibody. For example, fusion proteins containing beta-galactosidase may be purified by affinity chromatography using an anti-beta-galactosidase antibody column (Ullman, Gene. 29:27-31 (1984)). Similarly, fusion proteins containing maltose binding protein may be purified by affinity chromatography using a column containing cross-linked amylose; see Guan, European Patent Application 286,239.

The protein may occur at the amino-terminal or the carboxy-terminal side of the cleavage site. Optionally, the DNA that encodes the fusion protein is engineered so that the fusion protein contains a cleavable site between the protein and the fusion partner. Both chemical and enzymatic cleavable sites are known in the art. Suitable examples of sites that are cleavable enzymatically include sites that are specifically recognized and cleaved by collagenase (Keil et al., FEBS Letters 56:292-296 (1975)); enterokinase Prickett, K. S. et al., Biotechniques 7:580-589 (1989); LaVallie et al., J. Biol. Chem. 268:23311-23317 (1993)); factor Xa (Nagai et al., Methods Enzymol. 153:461-481 (1987)); and thrombin (Eaton et al., Biochemistry 25:505 (1986) and Chang, J. Y. Eur. J. Biochem. 151:217-224 (1985)). Collagenase cleaves between proline and X in the sequence Pro-X-Gly-Pro wherein X is a neutral amino acid. Enterkinase cleaves after lysine in the sequence Asp-Asp-Asp-Asp-Lys. Factor Xa cleaves after arginine in the sequence Ile-Glu or Asp-Gly-Arg. Thrombin cleaves between arginine and glycine in the sequence Arg-Gly-Ser-Pro.

Specific chemical cleavage agents are also known. For examples, cyanogen bromide cleaves at methionine residues in proteins (Gross, E., Methods Enzymol. 11:238-255 (1967), hydroxylamine cleaves at Asn-Gly bonds (Bornstein, G. and Balian, G., J. Biol. Chem. 245:4854-4856 (1970), and by hydrolysis at low pH (Asp-Pro bonds are labile at low pH; Landon, M., Methods Enzymol. 47(E):145-149 (1977).

D. General Methods for Purification of Proteins

The recombinant protein is purified by methods known in the art. Such methods include affinity chromatography using specific antibodies. Alternatively, the recombinant protein may be purified using a combination of ion-exchange, size-exclusion, and hydrophobic interaction chromatography using methods known in the art. These and other suitable methods are described by Marston, “The Purification of Eukaryotic Proteins Expressed in E. coli” in DNA Cloning, D. M. Glover, Ed., Volume III, IRL Press Ltd., England, 1987; Guide to Protein Purification, Deutscher, M. P. (Ed.), Methods Enzymol. 182, Academic Press, Inc., New York (1990); Scopes, R. K. and Cantor, C. R. (Eds), Protein Purification (3d), Springer-Verlag, New York (1994); and by Sofer et al. in Biotechniques_{—}, 198-203 (1983).

### Materials and Specific Methods

Propagation of Lewis Lung Adenocarcinoma Tumors. C57 B1 mice (Charles River Breeding Laboratories) were provided free access to standard laboratory chow and water. To generate tumors, approximately 1.75×106 Lewis lung mouse carcinoma cells (LL/2, American Type Culture Collection, CRL 1642) were injected subcutaneously into 15 gm C57 B1 females, and the tumors allowed to grow for two weeks before passage. Under light pentobarbitol anesthesia (Membumal, 75 mg/kg body weight), a dorsal incision was made and approximately 3 mm3 of viable tumor cortex was implanted subcutaneously. Tumors were passaged at least three times prior to use. Animals were sacrificed by cervical dislocation, and tumors were removed and stored at −80° C. until use. All animal protocols were approved by the Animal Care and Use Committees of Cornell University Medical College. Cell Culture. LL/2 cells were grown in 25 cm2 plastic tissue culture flasks (Coming) under 5% CO2 in Dulbecco's Modified Eagles Medium (Mediatech) containing 10% fetal calf serum (Hyclone) and 0.01% penicillin/streptomycin (Gibco Laboratories). MDCK cells were grown under indentical conditions except in 5%fetal calf serum with no antibiotics. Cells were plated at a density of 10⁵ cells/ml and passaged upon reaching 80-90% confluence.

Immunoblots. Cell lysates were resolved by SDS-PAGE on 10% acrylamide, 0.27% bis-acrylamide gels (Laemmli, U. K., and M. Favre. 1973. J. Mol. Biol. 80: 575-599) and electrophoretically transferred to nitrocellulose paper in (40 mM Tris, 240 mM glycine, 20% ethanol, 0.2% SDS) transfer buffer (Towbin, H., T. Straehelin, and J. Gordon. 1979. Proc. Natl. Acad. Sci. USA. 76: 4350-4354). Transferred protein was incubated either with a.) 0.1 to 5.0 μg/ml of affinity-purified Anti-DdSev, a polyclonal antibody raised against Dictyostelium severin, b.) 0.1 to 5.0 μg/ml Anti-MSev, a polyclonal antibody raised against mammalian severin isolated from Lewis lung adenocarcinoma tumors, or c.) 4 μg/ml monoclonal antibody to gelsolin (Sigma). Immunoblots were developed with alkaline phosphatase-conjugated secondary antibody and BCIP-NBT (Promega) following the manufacturer's instructions.

Alternatively, cell lysates were prepared from cell cultures by scraping cells from a single culture dish, sedimenting at 700×g for 5 min. and dissolving the cell pellet in 200 ul of electrophoresis sample buffer (0.2% SDS, Tris, pH 8.5, bromphenol blue). SDS-PAGE was with 10% acrylamide and 0.27% bis-acrylamide following the protocol of Laemmli and Favre (1973). Gels were electorphoretically transferred overnight to nitrocellulose paper (Towbin et al., 1979). Blots were incubated in Tris-buffered saline containing 1% Tween (TBST) and either 5% blot-qualified bovine serum albumin (BSA) or 5% non-fat milk solids (Carnation) for 30-60 min. to block nonspecific binding sites. Blocking solution was replaced with primary Ab serum diluted 1:200 with 1% BSA-TBST for 1 hour, and washed with 1% BSA-TBST 3×10 min. Incubation with secondary Ab (alkaline phosphatase conjugated goat anti-rabbit Fab₂, Jackson Immunolabs) for 30 min, followed by washing in TBST 3×10 min. Bands were detected with BCIP NBT developer (Promega) following the manufacturer's instructions.

M30 protein, Annexin II and tyrosine phosphorylation were detected by Western immunoblot analysis against LLC cell lysates, (enriched fractions of M30 from LLC lysates), and biochemically extracted LLC cells. Cell fractions were resolved by SDS-PAGE on 10% acrylamide gels according to Laemmli and Favre (1973). Electrophoretic transfer to nitrocellulose paper was performed according to Towbin et al. (1979). Transferred blots were blocked with 1% Fraction V Bovine Serum Albumin (Sigma Chemicals) in TBST (50 mM Tris-HCl, pH7.4, 150 mM NaCl, 0.1% Tween) for 30 minutes. M30 was identified with Anti-M30 polyclonal antibody, raised against Dictyostelium p34. Annexin II was identified with a monoclonal antibody (Transduction Laboratories). Tyrosine phosphorylation was detected with a monoclonal Py-20 antibody (Santa Cruz Biologicals). Primary antibodies were diluted to 1:10,000 in 1% BSA+TBST for development by the ECL chemiluminescence system (Amersham) or 1:1000 in 1% BSA+TBST for development by the alkaline phosphatase Western Blue system (Promega) and incubated for 30 minutes at room temperature. Blots were washed three times, 5 min. each, in TBST. Secondary antibodies used were HRP-conjugated goat anti-rabbit Fab_{2′} (Jackson Labs) for the ECL system or alkaline phosphatase-conjugated donkey anti-rabbit Fab_{2′} for the Western Blue system. Secondary antibodies were diluted to 1:20,000 for ECL or 1:5,000 for Western Blue and incubated for 30 minutes in 1% BSA+TBST at room temperature. The blots were washed again 3 times, 5 min. each time, in TBST followed by a 1-5 min. development in ECL or a 2-10 min. development in Western Blue. ECL blots were exposed to X-ray film (Fuji) for 30 s to 10 minutes to visualize.

Actin Filament Severing Assays. Rabbit muscle F-actin was used as a substrate for M-severin. Fractions to be assayed for severing activity were added to 0.1 mg/ml F-actin in F-buffer (10 mM triethanolamine, pH 7, 0.2 mM dithiothreitol, 50 mM KCl, 2 mM MgCl2, 1 mM ATP) containing either 0.1 mM CaC12 (+Ca2+) or 2 mM EGTA (−Ca2+). Mixtures were incubated for 10 min. at 25° C. Aliquots (10 μl) of the reaction mixture were placed on parlodion, carbon-coated grids and stained for 1 min. with 0.2 μm filtered 1% uranyl acetate. Stained grids were blotted on the edge with filter paper, air-dried and viewed in a JEOL 2000 electron microscope at 80 kV accelerating voltage. To quantitate severing efficiency, mixtures resulting from severing assays were centrifuged at 50,000×g for 15 min. to differentially sediment intact actin filaments. Resulting supernatant (actin monomers+fragments) and pelleted (actin filament) fractions were resolved by SDS-PAGE, and the actin and severin content assayed by gel scanning densitometry (Hoeffer, San Francisco, Calif.).

Purification of Mammalian Severin. Isolation of mammalian severin from Lewis lung adenocarcinoma tumors followed the purification method previously established for Dictyostelium severin, (See Brown, S. S., K. Yamamoto, and J. A. Spudich. 1982. A 40,000-dalton protein from Dictyostelium discoideum affects assembly properties of actin in a Ca2+-dependent manner. J. Cell Biol. 93: 205-210; And Yamamoto, K., J. D. Pardee, J. Reidler, L. Stryer, and J. A. Spudich. 1982). Mechanism of interaction of Dictyostelium severin with actin filaments. J. Cell Biol. 95: 711-719. with slight modification. Tumor burdens of 15% to 20% of total body weight were excised, rinsed with 5 mM triethanolamine buffer, pH 7.5, and stored at 80° C. until use. All isolation steps were carried out at 4° C. or on ice. For each preparation, approximately 50 gms of tumor tissue was thawed, minced and added to 3 volumes (wt/vol) of cold Lysis Buffer (10 mM triethanolamine, pH 7.5, 60 mM sodium pyrophosphate, 30% (wt/vol) sucrose, and 0.4 mM dithiothreitol). Phenylmethylsulfonylfluoride in ethanol was added to a final concentration of 1 mM and the suspension was immediately sonicated on ice with 3×30 s bursts (Heat Systems W-220F sonicator at 30 MHz power). The cell lysate was centrifuged at 25,000×g for 30 min., and the supernatant fraction was recentrifuged at 150,000×g for 90 min. Total protein concentration was determined for the high speed supernatant fraction (See Brock, A. M., and J. D. Pardee. 1988. Cytoimmunofluorescent localization of severin in Dictyostelium amoebae. Dev. Biol. 128: 30-39), and the fraction diluted to 5 mg/ml with cold Lysis Buffer. Triethanolamine (1 M, pH 7.5) was added to obtain a final concentration of 50 mM. Solid ammonium sulfate was incrementally added to obtain 60% saturation at 0° C., and the mixture was stirred on ice for 1 hr. After centrifugation at 25,000×g for 30 min., the resulting supernatant fraction was brought to 80% saturation on ice with solid ammonium sulfate. The 80% ammonium sulfate pellet was collected by centrifugation at 25,000×g for 30 min., dissolved in 20 ml DEAE Buffer (2 mM triethanolamine, pH 7.5, 0.2 mM dithiothreitol, 0.005% NaN3) and dialyzed for 24 hr against 3×2 l. of DEAE Buffer containing 2 mM KCl. The dialyzed fraction was applied to a 1.5×15 cm DEAE cellulose column, (DE 52, Sigma) pre-equilibrated with DEAE Buffer containing 2 mM KCl. Bound protein was eluted at 5 ml/hr in 2.5 ml fractions with a 0-0.6 M KCl linear gradient. Severing activity eluted from 0.05 to 0.15 M KCl. Active fractions were dialyzed overnight against 2×1 l. HAP Buffer (10 mM KH2PO4, pH 6.7, 0.2 mM dithiothreitol, 0.005% NaN3). The dialyzed fraction was applied to a 1.0×14 cm hydroxylapatite column (Calbiochem) equilibrated with HAP Buffer. Bound protein was eluted at 5 ml/hr in 2.0 ml fractions with a 0-0.6 M KCl linear gradient. Purified M-severin eluted at approximately 0.3 M KCl and was stored on ice until use.

Antibodies. A rabbit polyclonal antibody raised against purified Dictyostelium severin (Brock, A. M., and J. D. Pardee. 1988. Dev. Biol. 128: 30-39) was isolated by chromatography through a Zeta Chrom 60 Disk (Cuno, Inc.). Severin-specific IgG (Anti-DdSev) was subsequently affinity-purified using purified Dictyostelium severin cross-linked to a CNBr-activated Sepharose 4B column, according to the methods of Johns, J. A., A. M. Brock, and J. D. Pardee. 1988. Cell Motility and the Cytoskeleton. 9: 205-218, hereby incorporated by reference). The antibody Anti-MSev was raised in rabbits by subcutaneous injection of purified M-severin from Lewis lung carcinoma tumors. Injection of 2 μg of protein in complete Freund's adjuvant at each of 6 dorsal sites was followed by an equivalent challenge inoculation after two weeks and bleedings at 2 week intervals. Positive sera was stored at −20° C. A monoclonal antibody to human plasma gelsolin showing specificity to an epitope on the 47 kD non-severing chymotryptic peptide (see Chaponnier, C., P. A. Janmey, and H. L. Yin. 1986. The actin filament-severing domain of plasma gelsolin. J. Cell Biol. 103: 1473-1481), was purchased from Sigma Chemical Co. (#G 4896).

Cytoimmunofluorescent Localization. LL/2 cells were grown on 15 mm diameter glass coverslips, rinsed with PBS (0.15 M NaCl, 0.015 M Na2HPO4, pH 7.4), fixed by immersion in −20° C. methanol for 10 min., held under PBS for 15 min., and blocked with PBS+1% BSA for 15 min. Coverslips were incubated for 60 min. at 25° C. with 2-3 μg/ml Anti-MSev, washed with PBS (3×10 min.), PBS+1% BSA (15 min.), and incubated with 1.8 μg/ml FITC-conjugated mouse anti-rabbit IgG, F(ab′)2 (Jackson ImmunoResearch) for 60 min. at 25° C. F-actin was stained with 0.33 μM rhodamine phalloidin (Molecular Probes) for 60 min. at 25° C. on parallel coverslips. Labeled cells were washed with PBS (3×10 min.) and coverslips mounted on glass slides with gelvatol [15% (w/v) polyvinyl alcohol (Airvol 205, Air Products and Chemicals, Inc.), 65% glycerol (v/v), 35% PBS (v/v)] containing 100 mg/ml 1,4 Diazabicyclo[2.2.2.] Octane (DABCO, Sigma) prior to viewing under a Nikon Microphot microscope.

Alternatively, cells were sterile plated directly onto 15 mm round glass coverslips in tissue culture well plates (Coming). For staining, coverslips were washed with phosphate buffered saline (PBS; 0.15 M Nacl, 0.015 M Na2HPO4, pH 7.4) for 15 minutes to remove unattached cells. Attached cells were fixed in prechilled −20° C. methanol for 10 minutes, followed by 1% BSA-PBS for 15 min. and PBS alone for 5 min. Sequential incubations were in primary antiserum diluted 1:100 in 1% BSA-PBS for 30 min and PBS for 3×10 min., donkey anti-rabbit Texas Red F(ab)2′ secondary antibody (Jackson ImmunoResearch) diluted 1:500 in 1% BSA-PBS for 30 min, PBS for 3×10 min and in distilled water for 5 min. Stained coverslips were blotted on edge and mounted on slides with Gelvator [15% (w/v) polyvinyl alcohol (Airvol 205, Air Products and chemicals, Inc.), 65% glycerol (v/v), 35% PBS (v/v)] containing 100 mg/ml 1,4 Diazabicyclo[2.2.2]Octane (DABCO, Sigma). Slides were dried overnight in the dark at room temperature and photographed with a Nikon epifluorescence microscope using Kodak Ektachrome ASA 400 film.

For M-30 and AnnexinII: Cultures of LLC cells were plated at a concentration of approximately 10⁵ cells/ml on glass coverslips that were precleaned with acetone and briefly flamed. Cells were grown for 1-2 days in DMEM+10% FCS at 37C under 5% CO2 until cells adhered and spread. A second precleaned glass coverslip was then floated on top of the culture and cells allowed to grow another 24 hr. or until well-spread. The coverslip sandwich was separated by forceps, quickly dipped 5× in PBS(150 mM NaCl, 150 mM NaHPO_{4,} pH 7.5, 0.005% sodium azide) and fixed in 20C methanol for 15 minutes. Both coverslips yielded spread cells. Following fixation, coverslips were briefly blotted on edge and rapidly transferred through 3 washes of PBS, each lasting 3 min. Coverslips were placed on parafilm in a humidity chamber. One coverslip was reserved for preabsorption of each secondary antibody. Cells were blocked with 1.5% Normal Goat Serum (Vector) in PBS for 20 minutes, incubated with primary antibodies (anti-M30/Annexin II serum, Anti-Annexin II, Anti-Dictyostelium p34, or Py-20) diluted 1:50 in PBS for 30 min, and washed 3×10 ml in PBS for three min. in each wash. Controls were incubated in preimmune rabbit serum diluted 1:50 in blocking solution. Secondary antibodies (FITC conjugated goat anti-rabbit FAB_{2′}, Jackson Immunoresearch) for M30/Annexin II or Texas Red conjugated goat anti-mouse FAB_{2′}, Jackson Immunoresearch for Py20 were diluted 1:600 in blocking solution and preabsorbed in the dark against PBS hydrated, fixed cells on a coverslip for 1 hour. Preabsorbed secondary antibody was applied to cells for 30 min. protected from light, and cells were washed 3 times, 3 min. each in PBS. To stain for actin filaments a 1:100 dilution of rhodamine phalloidin (Sigma) was added for 30 min. in the dark without prior blocking. Coverslips were dipped in deionized water 5× and mounted on a glass slide in gelvatol heated to 37C before examination by confocal microscopy.

Selective Biochemical Extraction of LLC Cells. LLC cells cultured in DMEM containing 10% FCS were rinsed with PBS and harvested in PBS by gentle agitation. Cells were pelleted by centrifugation at 1000×g for 10 min., washed 3 times in PBS and recentrifuged. The cell pellet volume was measured and a 3×volume of the appropriate extraction buffer was added to the pellet. All lysis buffers were prechilled on ice. Physiologic salt extraction buffer contained 10 mM triethanolamine, pH 7.0, 100 mM KCl, 1 mM MgCl₂, and a cocktail of proteolysis inhibitors added immediately before use (0.1 mM phenylmethylsulfonyl fluoride (Sigma), 10 ug/ml Leupeptin, 10 ug/ml Apoprotinin, and 0.1 mM Vanadium). Sucrose extraction buffer contained 30% sucrose, 10 mM triethanolamine, pH 7.5, 1 mM EDTA, and 0.4 mM dithiothreitol plus inhibitors. Triton extraction buffer contained 140 mM KCl, 10 mM triethanolamine, pH 7.0, 1 mM DTT, 0.5 mM MgCl, 1% Triton X-100 and inhibitors. Triton X-100 extraction was carried out on ice for 30 minutes. For the sucrose and physiologic salt extractions, Dounce homogenization on ice or brief sonication (several 2 second bursts at 30 MHz power, Heat Systems 220F Cell Disrupter) was used to disrupt cells. Cell disruption was monitored by phase contrast microscopy. The resulting lysates were centrifuged in a tabletop microfuge at 14,000 rpm for 15 minutes at 4C and the pellet reserved. The supernatant fraction was recentrifuged at 100,000×g for 30 minutes at 4° C. An equal volume of SDS gel solubilization buffer (Laemmli and Favre,ibid.) was added to each supernatant and a 2×supernatant volume of sample buffer was added to each pellet. Samples were boiled for 10 minutes, and 3 ul were loaded per lane on a minigel for SDS-PAGE.

In Situ Triton Cytoskeleton Preparation. LLC cells were cultured on coverslips as described for cytoimmunofluorescence. Prior to methanol fixation, coverslips were prechilled on ice. Cold coverslips were incubated for exactly 10 minutes on ice in 0.5 ml of Triton extraction buffer containing protease inhibitors as described for biochemical extractions. Extracted cells were quickly dipped 5× in ice-cold PBS and plunged into −20C methanol for 15 minutes. Cells were then stained as described in cytoimmunofluorescence. One Triton treated coverslip was reserved for preabsorption of each secondary antibody used.

Confocal Microscopy. LL/2 cells were treated as described for cytoimmunofluorescent staining and 1 μm thick optical sections were examined with a Sarastro 2000 confocal laser scanning microscope (Molecular Dynamics, Sunnyvale, Calif.) using Image Space software.

Alternatively, for M-30 and Annexin II determinations: A through-focus series of optical sections were obtained using a Sarastro laser confocal microscope, fitted with a computer-controlled stepping motor (0.2 um step size) and a 100× Plan Acromat Nikon objective. Images were collected from a region in the center of the field to limit spherical aberration. The field was illuminated with a scanning argon laser set for a wavelength of 488 nm for FITC, 514 nm for Texas Red and rhodamine phalloidin, or left open for dual image scanning. A primary dichroic beamsplitter of 510 nm and an aperture of 100 um were used. Digital images were acquired with a photomultiplier tube fronted with filters of 530 nm for FITC and 610 nm for Texas Red or rhodamine phalloidin. The FITC channel was read at a power of 750W while the red channel was set to 900W. When dual images were required, a 565 DRLP secondary beam splitter was used. Images were acquired, stored, and analyzed by a Personal Iris computer (Silicon Graphics) running Imagespace software (Molecular Dynamics).

RNA Isolation and Northern Blot Hybridization. RNA used in Northern analysis was isolated from cultured P19, LL/2 and MDCK cells using the RNeasy Total RNA Kit (Qiagen, Chatsworth, Calif.) according to the manufacturer's instructions. Approximately 50 μg per well of RNA was subjected to electrophoresis through a 1.2% agarose gel containing 2.2 M formaldehyde. RNA was transferred overnight to a positively charged nylon membrane (Boehringer Mannheim, Indianapolis, Ind.) by passive transfer in 20×SSC [1×SSC is 150 mM NaCl, 15 mM sodium citrate (pH 7.0)]. The insert from clone 10c-1 (0.65 kb) was purified from a 1% agarose gel using the QIAEX II Gel Extraction Kit (Qiagen), labeled with [alpha-32P] dCTP using the Random Primed DNA Labeling Kit (Boehringer Mannheim), and was used as a probe. Hybridization was performed in 50% formamide, 5×SSPE [1×SSPE is 0.18M NaCl, 1 mM EDTA, 10 mM NaH2PO4 (pH 7.5)], 0.2% SDS, 5×Denhardt's (39), and 100 μg/ml denatured salmon sperm DNA at 42° C. overnight. The hybridized membrane was rinsed twice at room temperature in 2×SSC/0.1% SDS, and then washed twice at 42° C. in 0.5×SSC/0.1% SDS for 30 min. The Membrane was exposed to a phosphor screen (Molecular Dynamics, Sunnyvale, Calif.) and images of the original radioactive samples were produced with a PhosphorImager (Molecular Dynamics). The data was analyzed using Molecular Dynamics ImageQuant software version 3.0.

Immunohistochemistry. Paraffin embedded surgical sections of a moderately differentiated adenocarcinoma of the large bowel were sectioned and stained for H-severin or M-30 with a Vectastain Elite ABC Kit (Vector Laboratories) using biotinylated anti-rabbit IgG as the secondary antibody with peroxidase substrate. Sections were deparaffinized, hydrated through an alcohol series, blocked with rabbit serum, incubated with primary antibodies against either purified Dictyostelium discoideum severin, or D. discoideum 30 KDa Protein; or M-severin isolated from Lewis lung carcinoma tumors from C57 mice. Primary antibodies were used at 1:200 for D.d. severin and M-30, or at 1:50 for LL2 M-severin. Secondary antibody was at 1:200. M-severin stained sections were counterstained with hematoxylin. M-30 stained sections were counterstained with hemotoxylin, viewed on a Nikon epifluorescence microscope and photographed with Kodachrome ASA 25 film.

Other Methods. Tris-glycinate SDS-PAGE was performed according to Laemmli and Favre Laemmli, U. K., and M. Favre (1973). using 1 mm thick slab gels. Molecular weight standards (Pharmacia) were phosphorylase b (94 kDa), bovine serum albumin (68 kDa), ovalbumin (43 kDa), carbonic anhydrase (30 kDa), soybean trypsin inhibitor (20 kDa) and a-lactalbumin (14 kDa). Protein concentrations were measured by the method of Bradford (40) using bovine plasma gamma globin as a standard. Gels for Western blots were stained with Coomassie Brilliant Blue G250. Free Ca2+ concentration was calculated using a Kd for Ca2+ EGTA of 2×10⁻⁷M.

### Specific Examples

1. M-Severin

Immunologic Detection of Severin in Lewis Lung Carcinoma Tumors. The requirement for cell migration in epithelial malignancy prompted a survey for severin in LL/2 cell Lewis lung carcinoma tumors. An antibody raised against Dictyostelium severin (Anti-DdSev) specifically detected a 40 kDa protein in both Dictyostelium and tumor cell lysates. Antibody avidity was 1000-fold greater for Dictyostelium severin, with positive Western blots obtained with 1 ng/ml Anti-DdSev compared to 1 μg/ml for the 4 kDa protein in tumor lysates.

Isolation of Severin from Tumors. To establish functional identity, the 40 kDa tumor protein was purified from LLC tumors by methods previously established to isolate severin from Dictyostelium amoebae (See Brown, S. S., K. Yamamoto, and J. A. Spudich. 1982. A 40,000-dalton protein from Dictyostelium discoideum affects assembly properties of actin in a Ca2+-dependent manner. J. Cell Biol. 93: 205-210; Yamamoto, K., J. D. Pardee, J. Reidler, L. Stryer, and J. A. Spudich. 1982. Mechanism of interaction of Dictyostelium severin with actin filaments. J. Cell Biol. 95: 711-719).

Isolation utilized ammonium sulfate fractionation of a clarified tumor lysate followed by DEAE and HAP chromatography. Purification was followed by both Ca2+-activated actin filament severing activity and immunoblots with Anti-DdSev. Final HAP chromatography produced a purified 40 kDa protein with Ca2+-activated severing activity and cross-reactivity to Anti-DdSev. The average yield of severin was 0.36 mg per 50 gms of tumor, representing 0.03% of total lysate protein. Like Dictyostelium severin, the tumor protein was completely soluble in 80% ammonium sulfate and eluted from HAP in 0.3 M KCl to give a pure product. However, the isolated mammalian severin was not biochemically identical to Dictyostelium severin, since M-severin showed a moderate affinity to DEAE at pH 7.5 compared to no affinity for Dictyostelium severin.

Functional Activity of M-severin. The actin filament fragmenting activity of purified tumor-derived severin was assayed by electron microscopy and differential sedimentation of various stoichiometric mixtures of severin and F-actin in the presence or absence of 50 μM Ca2+. Actin filaments remained intact in severin:actin mixtures in the absence of Ca2+ (presence of 2 mM EGTA), but were rapidly fragmented upon addition of Ca2+. Like Dictyostelium severin, increased ratios of M-severin to F-actin produced shorter fragments. At 1:100 M-severin:actin, fragment length averaged 30 nm, compared to an average length of 10.5 nm for 1:20 severin:actin. Fragment lengths corresponded to an average of 130 G-actin subunits in 1:100 fragments and 28 subunits in 1:20 fragments, indicating a stoichiometric rather than catalytic fragmenting activity by M-severin. Isolated M-severin did not induce coalignment, bundling or cross-linking of actin filaments, suggesting an exclusive fragmentation and capping activity.

To quantitate severing function, increasing ratios of severin:actin were sedimented at 50,000×g for 15 min. to separate short fragments from long fragments and filaments. Gel electrophoresis of separated filaments and fragments confirmed that M-severin action mimicked that of Dictyostelium severin, with enhanced fragmentation at higher ratios of severin: F-actin. Based on close similarities in size, immunologic cross-reactivity, purification properties and functional activity, M-severin has been identified as the mammalian homolog of Dictyostelium severin.

Selective Expression of M-Severin in Transformed Tissues. M-severin expression was compared in normal and transformed tissues. M-severin protein was not detected in normal skeletal muscle, liver, or lung taken from tumor-bearing animals. Since Lewis lung carcinoma tumors derive from pulmonary epithelium, it was of considerable interest to directly assay LLC tumors and normal lung tissue from the same animal for M-severin expression. Tumors showed extensive expression of M-severin in both the proliferating tumor cortex and necrotic core, while normal lung showed no cross-reactivity with Anti-DdSev, suggesting M-severin induction in neoplastic C57 B1 mouse lung epithelium.

Comparative Expression of M-Severin and Gelsolin in Normal and Transformed Epithelial Cells. Since severin and gelsolin both function as actin filament severing proteins, severin and gelsolin expression patterns were compared in normal lung and LLC tumors. To maximize sensitivity and specificity, a polyclonal antibody (Anti-MSev) was raised against purified M-severin isolated from mouse tumors. High levels of gelsolin were detected in normal lung lysates together with minute amounts of M-severin. Because highly motile fibroblasts, macrophages and neutrophils in pulmonary connective tissue contain M-severin, pneumocytes comprising the predominant lung epithelial cell type are not likely to contain the protein. M-severin is immunologically distinct from the N-terminal severin-like domain of gelsolin as evidenced by the lack of cross-reactivity between the Anti-MSev antibody and gelsolin in lung. The appearance of M-severin in transformed tissues cannot be ascribed to a proteolytic breakdown product of gelsolin. In transformed LL/2 tumor cells, expression of M-severin corresponded to a complete loss of gelsolin. We therefore posit that M-severin replaces gelsolin during epithelial cell transformation.

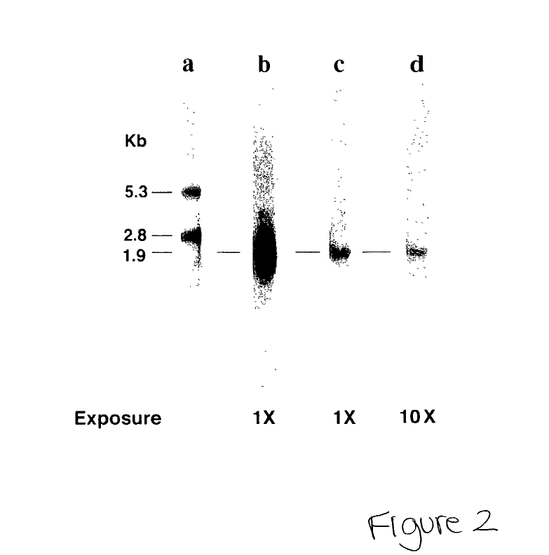

Anti M-sev was also used to clone a partial length M-severin cDNA from a P19 carcinoembryonic cell library (Stratagene). The clone, 10c-1 contained an 136 amino acid sequence with 48% homology to Dictyostelium severin. See FIG. 1. The clone allowed an analysis of the expression of M-severin mRNA in 3 epithelial cell lines (MDCK, LL/2 and P19 cells) having different metastatic potentials. Quantitation of blots by phosphoimaging (Molecular Dynamics) showed that highly metastatic P19 carcinoembryonic cells expressed approximately 10× as much M-severin mRNA as weakly metastatic LL/2 cells, which in turn showed 7× more mRNA than cultured MDCK cells (FIG. 2). M-severin expression in MDCK cell lines was approximately 70-fold less than in P19 cells. The low, but detectable, basal level of M-severin mRNA expression in MDCK cell cultures may reflect the partially transformed immortalized state of these cells. M-severin is consequently not exclusive to LL/2 cells, but is expressed in 3 different transformed epithelial cell types. Furthermore, M-severin messenger RNA expression showed a strong positive correlation with the metastatic potential of the cell line analysed.

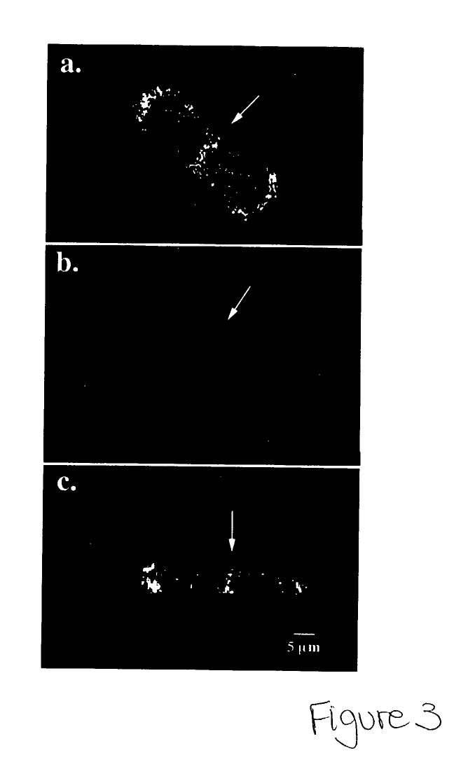

Localization of M-Severin. The intracellular location of M-severin in the actin cytoskeleton of dividing, migratory LL/2 cells was ascertained by confocal microscopy (FIG. 3). In actively dividing cells, M-severin was concentrated in the cleavage furrow and extending cell cortex distal to the furrow (FIG. 3a), and colocalized with F-actin (FIG. 3b). A vertical section through the dividing cell pair shown in FIG. 3a clearly shows high concentrations of M-severin at the leading cell edges and in the cleavage furrow (FIG. 3c). Mammalian severin and F-actin appear colocalized and concentrated in areas actively undergoing actin cytoskeleton rearrangements, consistent with severin localization in Dictyostelium Brock, A. M., and J. D. Pardee. 1988. Cytoimmunofluorescent localization of severin in Dictyostelium amoebae. Dev. Biol. 128: 30-39.

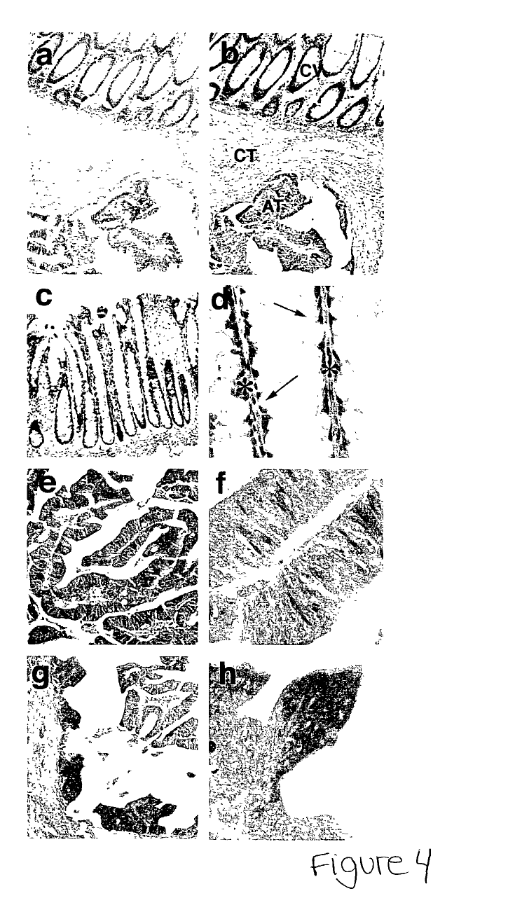

Specific Expression of H-severin in Invasive Colon Adenocarcinoma. That H-severin is expressed in epithelial carcinomas, but not normal epithelium, was demonstrated by immunohistochemical staining of adenocarcinomas of the colon (FIG. 4a, b) from 2 different patients. Sections through a surgically resected colon showed that H-severin was not expressed in normal colon epithelium (FIGS. 4c, 4d, arrows) from the cancer patient. Epithelial cells of normal colonic villi showed no severin staining (4d, arrows), while motile connective fibroblasts of the lamina propria underlaying the epithelium contained H-severin (FIG. 4d, *).

In moderately differentiated adenocarcinoma from the same patient, H-severin was abundantly expressed in transformed epithelial cells comprising the tumor (FIG. 4e, f). Furthermore, advanced stages of undifferentiated adenocarcinoma existing adjacent to moderately differentiated adenocarcinoma heavily expressed H-severin (FIG. 4g, arrow, 4h) suggesting that the extent of H-severin expression marks advancing stages of epithelial transformation. The striking up-regulation of H-severin in invasive carcinoma follows the paradigm of H-severin expression in motile, dividing cells documented in cell culture, and portends a significant potential use of H-severin as a marker for stage-specific diagnosis of carcinoma tumors. Similar detection of epithelial transformation by a H-severin marker has been observed in colon polyps and mammary ductal carcinoma.

2. M-30

Actin Cytoskeleton Rearrangement by Dictyostelium 30 kDa Protein.

Cell migration and division are absolute requirements for a wide variety of cell and tissue functions, including tumor cell metastasis. The actin network rearrangments essential for pseudopod extension and formation of the cleavage -furrow are induced by actin-associated cytoskeletal proteins. As isolated from Dictyostelium amoebae, the 30 kDa protein is a Ca⁺² regulated F-actin crosslinking factor (Fechheimer and Taylor, Fechheimer, 1987; Johns, et al., 1988) with a relative molecular mass of 34 kD that induces the formation of tightly registered and coaligned bundles of actin filaments. Each filament within the bundle is uniformly polarized. Opposite bundle ends contain either “barbed” rapidly growing or “pointed” slow growing actin filament ends. Consequently, these structures have the potential to grow unidirectionally, depending on the presence of pointed or barbed end actin capping factors, a property likely to be utilized during cell extension. By decreasing free Ca⁺² through the intracellular range from 10⁻⁵ M to 10⁻⁷ M, progressively larger bundles form rendering the 30 kDa protein under Ca⁺² control in the physiologic range of intracellular Ca⁺² concentrations.

An Ortholog of 30 kDa Protein is Expressed in Motile and Transformed Mammalian Cells. Epithelially derived Lewis lung carcinoma (LL/2) cells and P19 embryonic carcinoma cells both heavily expressed M-30 protein, while it was completely absent in confluent monolayers of MDCK epithelial cells. The ortholog displayed a slightly larger size (Mᵣ=35 kDa) compared to the Dictyostelium protein (34 kDa). We have named the mammalian protein M-30 and extended its observation to epithelial cells undergoing cell migration or transformation. In motile fibroblasts, M-30 is colocalized with F-actin at the leading cell edge and in stress fibers. In parallel cytoimmunofluorescent experiments with transformed epithelial LL/2 cells, M-30 was similarly restricted to the actin cytoskeleton of the extending cell margins and to the cleavage farrow of actively dividing cells. M-30 may therefore participate in cytokinesis as well as cell migration in transformed epithelial cells.

The paradigm of selective expression of M-30 protein only in motile cell populations was further substantiated by the absence of the protein in tissues composed primarily of sessile (non-migratory) cells. M-30 was absent from muscle, lung, liver, aorta and confluent MDCK epithelial cells by immunoblot assays. M-30 was also not detected in brain or skeletal muscle lysates from mouse, rabbit and chicken. Because M-30 was absent from non-motile cells, we conclude that M-30 is selectively expressed in migratory and dividing cell types, including transformed epithelial cells.

Selective Expression of M-30 in Migratory Cells from LL-2 Tumors.

The selective expression of M-30 protein in transformed epithelium cell lines led us to ask if M-30 expression could be correlated with the known invasive potential of LL/2 tumor cells. LL/2 cells were subcutaneously injected into C57 mice and tumors grown at the site of injection. LL/2 subcutaneous tumor produced in this manner are known to have low invasive and metastatic potential. Western blots showed that M-30 protein was significantly down-regulated in these low metastatic potential tumors. Excised, whole subcutaneous tumors were subsequently placed into cell culture medium containing 10% fetal calf serum. Within 24 hours of culture in the presence of serum, tumor cells migrated away from the tumor mass, and M-30 became simultaneously expressed in these serum-stimulated LL/2 tumor explants. As LL/2 tumor cells gained a motile phenotype, M30 became expressed. Serum stimulated LL/2 cells expressing M-30 showed high metastatic potential, as evidenced by the proliferation of liver and lung metastatses in CR57 mice following splenic injection of tumor explant cells. Consequently, enhanced cell motility together with heightened metastatic potential correlated with M-30 expression in LL/2 tumors.

Inverse Coordinate Expression of M-30 and E-cadherin During Epithelial Monolayer Formation.

The detection of M-30 protein solely in motile cells raises questions about the fate of M-30 expression in epithelial cells undergoing loss of motility, and how the expression of an actin motility regulator such as M-30 affects the expression and positioning of E-cadherin in epithelium. The expression pattern of M-30 and E-cadherin was assayed in MDCK cells progressing toward cell contact and formation of epithelial monolayers. Single cells in sparsely populated subconfluent cultures are highly motile, with abundant expression of M-30 and an absence of E-cadherin.

As motile cells contact one another E-cadherin expression becomes stimulated, resulting in the production of E-cadherin containing vesicles accumulating at the site of cell-cell contact. M-30 remains expressed throughout the process of initial cell contact. Freely motile edges of connected migratory cells are free of E-cadherin but retain M-30.

By 48 h in culture, individual cells formed small monolayer colonies. In new colonies, E-cadherin localized specifically in central punctate vesicles and at the plasma membrane in regions of cell-cell contact. Free cell margins contained high concentrations of M-30. Overall expression of M-30 gradually decreased as cells became incorporated into the nascent monolayer. Cells bordering the monolayer contained M-30 throughout the cytoplasm, with E-cadherin restricted to cell-cell contacts. Cells trapped within the monolayer showed E-cadherin concentrated at contact areas and degradation of M-30. By 72 h. in culture, mature MDCK monolayers were completely void of M-30. These observations indicate an opposing expression pattern for M-30 and E-cadherin during monolayer formation. M-30 expressed in subconfluent, motile cells rapidly disappears as E-cadherin is inserted into cell-cell contacts in maturing monolayers.

Epithelium Wounding Elicits Expression of M-30.

To determine if M-30 protein and E-cadherin are reversibly regulated by cell-cell attachments, confluent monolayers were physically scraped to create a strip “wound” within the epithelial sheet. Cytoimmunofluorescent staining of cell sheets for M30 and E-cadherin was performed on successive days following wounding. Within 24 hr of wounding, E-cadherin is completely lost from the cell cortex bordering the wound. Concommitant with E-cadherin loss, M-30 is rapidly expressed throughout border cells. The interior of the monolayer remains free of 30 kDa protein, and interior cell-cell junctions maintain E-cadherin. By 48 h post-wounding, border cell proliferation and migration into the wound is accompanied by abundant 30 kDa expression. M-30 appeared to be most heavily concentrated in the cell cortex, suggesting assembly of actin bundles at cell edges. In contrast, E-cadherin was not expressed in actively dividing and migrating cells closing the wound. Loss of cell-cell contacts along the wound edge therefore simultaneously induced expression of M-30 and degradation of E-cadherin. When wounded cell sheets “healed” by regrowth to full confluency, expression of 30 kD protein ceased, and E-cadherin became re-expressed. M-30 is an actin regulatory protein is reversibly expressed by gain and loss of cell-cell contacts. Intact epithelia therefore appear not to contain the full complement of cytoskeletal proteins required for cell motility, but retain the capacity to express necessary motility components when cell-cell contacts are lost.

In Situ Expression of M-30 Protein in Advancing Stages of Human Colon Adenocarcinoma.

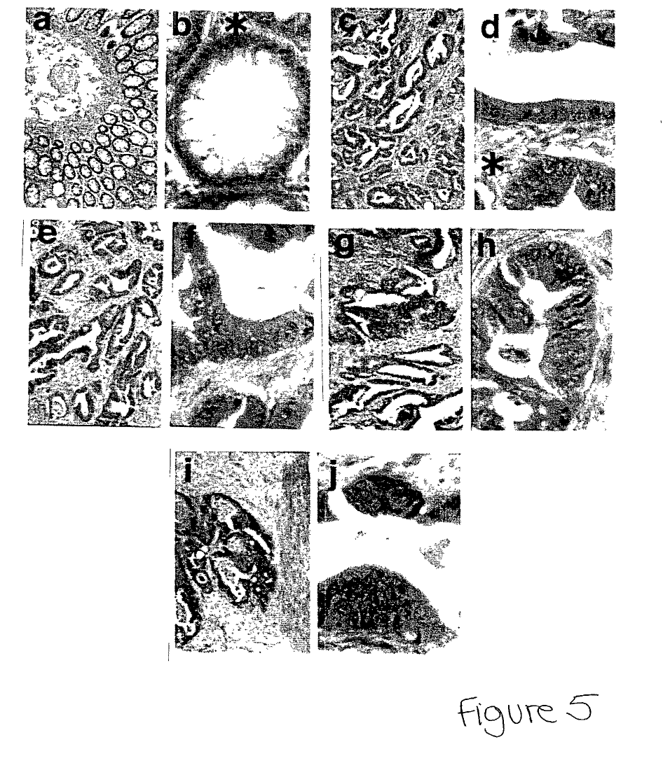

The regulated expression of M-30 protein in motile and transformed epithelial cells forces the question of whether the protein is a diagnostic marker for transformation, invasion or metastasis of carcinoma tumors. Here we present the initial evidence that human M-30 is expressed in human colon adenocarcinomas, but not normal colon (FIG. 5). Immunocytochemistry on serial sections of resected colon adenocarcinoma tumors from four patients showed that the human form of M-30 (i.e. H-30) was expressed in well-differentiated, moderately differentiated and undifferentiated adenocarcinoma tumors, but was not expressed in normal colon epithelium from the same patient. The human form of M-30 (i.e. H-30) was absent from normal epithelial cells lining colonic villi (FIGS. 5a, 5b). Both well-differentiated (FIGS. 5c, 5d) and moderately differentiated tumors (FIGS. 5e, 5f) from the same patient expressed significant amounts of the human form of M-30 (i.e. H-30). More advanced stages of undifferentiated adenocarcinoma showed heavy expression of the human form of M-30 (i.e. H-30) (FIG. 5g, arrow, 5h), suggesting that levels of H-30 expression mark advancing stages of epithelial carcinoma. In all cases, motile connective tissue fibroblasts of the lamina propria underlaying the epithelium stained lightly for H-30 (5b,d*). A similar upregulation of H-30 has been observed in transformed colon polyps and mammary ductal carcinoma. The dramatic expression of H-30 in invasive carcinoma follows the paradigm of H-30 function in motile, dividing cells documented in cell culture, and portends a potential use of H-30 as a marker for stage-specific diagnosis of carcinoma tumors.

3. Tyrosine Phosphorylated M-30 Protein and Annexin II:

LLC cells express Tyrosine Phosphorylated M30 Protein and Annexin II.

Lysates from interphase Lewis Lung carcinoma cells (LLC) were immunoblotted with antibody specific for M30, phosphotyrosine, or Annexin II using ECL chemiluminescence. M30 and Annexin II showed relative mobilities (Mr) centered at 34, and 36 kD respectively. M30 also cross-reacted strongly with antityrosine phosphate. While M30 appeared as the dominant phosphorylated protein in LLC lysates, other phosphorylated proteins appeared at longer ECL exposure times, however Annexin II never appeared as a tyrosine phosphorylated constituent. Expression of tyrosine phosphorylated M30 and Annexin II prompted a definition of their cytoskeletal locations in discrete structures supporting the enhanced proliferation and migratory states of transformed epithelial cells.

M30 and Annexin II segregate to distinct cytoskeletal domains in interphase LLC cells.

Confocal cytoimmunofluorescent localization of M30 protein was performed on interphase LLC cells undergoing spreading and migration. A horizontal 0.1 um optical cross section revealed punctate staining on the ventral plasma membrane, especially within the lamellapodia extending from the cell. The measured size of these ring structures, 1um, as well as their location suggests an association with adhesion plaques (Nuckolls, et. al., 1990). Further staining on the floor appeared in fibrous strands that weave into a network, indicative of the actin cytoskeleton, with which M30 is known to associate. (Johns, et. al., 1988, Fechheimer et. al., 1984) In a cross sectional view M30 localized to the dorsal cell surface, following the contours of the cell and suggesting association with the entire cortical actin network. A compartment was also localized deep within the cytoplasm surrounding the nucleus. Another cell chosen for its motile state showed the same focal adhesions on the floor, the cortical actin network, but also strongest localization at the leading front of the cell where the majority of actin rearrangement is actively occurring.

Annexin II in contrast showed exclusive compartmentalization to the perimeter of the cell, consistent with localization at the plasma membrane. In the horizontal cross-section., it was notably absent from the ventral plasma membrane and very little appeared within the cytoplasm (<1%). On vertical cross-section. Annexin appeared restricted to the lateral plasma membrane, failing to stain either the dorsal or ventral cell surfaces.

To confirm the association of M-30 with both phosphotyrosine and actin, co-localization was performed. Tyrosine in red and M30 in green combines to make yellow when colocalized. A computer generated histogram indicated 80% colocalization of the two signals and by immunofluorescence, colocalization especially occurred in the focal adhesions on the cell floor in the lammellapodia, confirming the identification of M30 as a major tyrosine phosphorylated species in the cell. Analysis of actin-M30 staining revealed 70% colocalization and clearly demonstrates association within the actin cytoskeleton and focal adhesion plaques.

Segregation of M30 to the Actin Cytoskeleton and Annexin II to the Plasma Membrane by Differential Biochemical Extraction.

Substrate-adhered LLC cells were gently lysed into three different biochemical buffers, insoluble components separated by sedimentation, and the resulting fractions immunoblotted for M30, Annexin II, and phosphotyrosine. All fractions were standardized for equal amounts of protein. Physiologic salt buffer, which extracts soluble cytoplasmic components, showed that neither M-30 nor Annexin II was freely soluble in the cytoplasm, but associated with insoluble cytoskeleton and membrane-associated macromolecular structures. Lysis in sucrose buffer solubilizes the actin cytoskeleton, but leaves organelles, membranes, and integral membrane proteins largely intact and insoluble (Moring et. al., 1975). Tyrosine phosphorylated M30 and Annexin II are solubilized by sucrose, indicating that they are not integral membrane proteins in either organelles or the plasma membrane. A third buffer system containing Triton X-100 was used to differentially solubilize the plasma membrane while keeping intact the internal actin cytoskeleton with its associated focal adhesion sites and stress fibers (Ben Ze'ev et. al., 1979). Following Triton extraction M30 segregated exclusively with the internal actin cytoskeleton in the Triton pellet while Annexin II appeared in the supernatant, indicating a presence only in the plasma membrane. Due to the identical electrophoretic movement, band appearance, and complete segregation with M30 in each of the three lysis buffers, tyrosine phosphorylation of M30 was confirmed.

To further explore Triton X-100 localization, extracted cells were immunofluorescence stained for M30, Annexin II, tyrosine phosphorylation, and Actin. Localization of M30, actin, and tyrosine phosphorylation were all confined to distinctive rings and fibers abundant in the cellular extensions and cell floor as members of the cytoskeleton. Triton extraction actually improved microscopic resolution and allowed identification of these rings as annuli surrounding focal contact sites (Davies et. al., 1994). Annexin II staining was absent in triton extracted cells as predicted by the electrophoresis data.

Identification of Tyr-phosphorylated M30 as a Component of the Cleavage Furrow.

A dividing cell stained with Anti-M30 showed some localization to the internal cytoskeleton focal adhesion sites as in interphase cells. At early cytokinesis, however, M30 shifted to the proximity of the cleavage furrow, apparently recruited from the cell adhesion plaques, most of which disappear from the cell floor. Consequently, these cells often become less attached to the substrate, forming a rounded shape. After leaving the floor, M30 surrounded the daughter nuclei and then progressed towards and extended through the furrow itself. A cross section taken directly through the cleavage furrow shows an abundance of M30, in strands and circular structures, indicating a role in formation of the cleavage furrow. In later stages, M30 appears throughout the contracted furrow, filling the entire space from floor to dome.

Annexin II, conversely, appears to play a more passive role in the cleavage furrow. During the process of formation and contraction, Annexin II is localized along the entire lateral plasma membrane, without rearrangement or change in distribution. As the cleavage furrow contracts, Annexin II becomes localized around the perimeter of the entire ring, but this may simply result from being pulled from the existing membrane. Finally, with the completion of division, Annexin II remained in the same lateral distribution as in interphase.

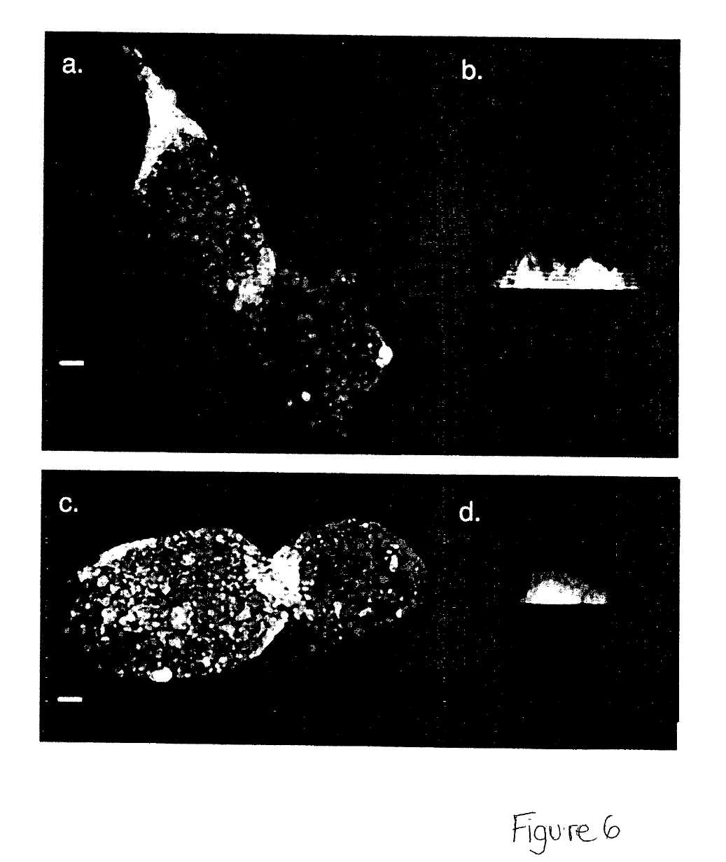

To confirm the presence of actin and M30 together in the cleavage furrow, a double staining was performed on dividing LLC cells. The horizontal cross section at the cell floor (FIG. 6a) shows strong colocalization apparent in the furrow. A significant portion of actin remains throughout the cell in fibrous strands and at the cortical actin network, however a majority of the M30 appears redistributed to the cleavage furrow with a minority on the floor and cortex. A vertical cross section through the furrow (FIG. 6b) shows that both the cortical actin network and internal actin cytoskeleton lie within the cleavage furrow however the majority of M30 appears to segregate to the internal cytoskeleton, substantiating its active role in furrow formation and contracture. By histogram 80% of the signal colocalized between these two proteins.

M30 was also doubly stained with Anti-tyrosine phosphorylation antibody to detect if the M30 within the cleavage furrow was tyrosine phosphorylated. An optical horizontal section (FIG. 6c.) demonstrated that M30 staining both in the redistributing portion as well as in the cleavage furrow to be colocalized with tyrosine phosphorylation to a high extent (72% by computer quantified analysis). A vertical cross-section taken directly through the furrow revealed tyrosine phosphate and M30 extending from the floor to dome within the furrow. Redistribution of tyrosine phosphorylated M30 from focal contact sites to the dividing cell's cleavage furrow is therefore defined as a major feature of conversion to a replication phenotype.

### Interpretation of Examples

1. M-Severin Localization

The detection of a mammalian severin significantly broadens the occurrence of a protein previously presumed to be expressed only in Dictyostelium amoebae and Physarum slime molds (fragmin). Severin has traditionally been considered ancestral to gelsolin, the 80 kD F-actin fragmenting protein in mammalian cells, because of extensive sequence homology and because gelsolin is not expressed in Dictyostelium amoebae. Expression of a mammalian severin presents the case for evolutionary conservation of a distinct severin gene. The gene product shows strong immunologic and functional identity to Dictyostelium severin, and shares a common cellular location in the actin-rich cortex. However, M-severin does not derive from a proteolytic breakdown product of gelsolin, since antibodies specific for M-severin do not recognize gelsolin. Two other actin associated proteins, gCap 39, and with Mr's approximating M-severin have been described in mammalian cells, but do not function as F-actin fragmenting proteins. Based on sequence similarity, MCP, gCap39, Mbh1, gelsolin, villin and actin binding protein (ABP) all belong to a family of mammalian actin filament regulatory proteins evolved from a structural motif composed of 120-130 amino acids found in Dictyostelium severin. We propose that M-severin itself now be added to the family of actin-regulatory proteins expressed in mammalian cells, including mouse cells, and as H-severin in human cells.

Although severin has been implicated in Dictyostelium cell motility by its Ca2+-activated F-actin severing function (See Brown, S. S., K. Yamamoto, and J. A. Spudich. 1982. A 40,000-dalton protein from Dictyostelium discoideum affects assembly properties of actin in a Ca2+-dependent manner. J. Cell Biol. 93: 205-210; Yamamoto, K., J. D. Pardee, J. Reidler, L. Stryer, and J. A. Spudich. 1982. Mechanism of interaction of Dictyostelium severin with actin filaments. J. Cell Biol. 95: 711-719. and restricted localization to extending pseudopods Brock, A. M., and J. D. Pardee. 1988. Cytoimmunofluorescent localization of severin in Dictyostelium amoebae. Dev. Biol. 128: 30-39), the function of severin in migrating amoebae has not been determined. This is largely due to the inability to produce a non-motile phenotype in Dictyostelium mutants lacking severin. The precise function of actin fragmentation in highly motile transformed mammalian cells is also problematic because gelsolin, the only fragmentation protein found to date in epithelial cells, is almost completely downregulated during transformation, (Vandekerckhove, J., G. Bauw, K. Vancompernolle, B. Honore, and J. Celis. 1990. Comparative two-dimensional gel analysis and microsequencing identifies gelsolin as one of the most prominent down-regulated markers of transformed human fibroblast and epithelial cells. J. Cell Biol. 111: 95-102; Chaponnier, C., and G. Gabbiani. 1989. Gelsolin modulation in epithelial and stromal cells of mammary carcinoma. Amer. J. Pathol. 134: 597-6030.

In fact, a significant number of actin cytoskeleton proteins germane to cell migration and cytokinesis are extensively down-regulated in proliferating and migrating cancer cells, Tropomyosins, ABP, caldesmon and gelsolin are all substantially diminished or deleted. Especially puzzling has been the disappearance of gelsolin from highly motile transformed human fibroblasts, epithelial cells (See Vandekerckhove, J., G. Bauw, K. Vancompernolle, B. Honore, and J. Celis. 1990. Comparative two-dimensional gel analysis and microsequencing identifies gelsolin as one of the most prominent down-regulated markers of transformed human fibroblast and epithelial cells. J. Cell Biol. 111: 95-102) and human breast carcinoma tissue (Chaponnier, C., and G. Gabbiani. 1989. Gelsolin modulation in epithelial and stromal cells of mammary carcinoma. Amer. J. Pathol. 134: 597-603), because enhanced rates of cell migration are known to occur in fibroblasts overexpressing gelsolin (Cunningham, C. C., T. P. Stossel, and D. J. Kwiatkowski. 1991. Enhanced motility in NIH 3T3 fibroblasts that overexpress gelsolin. Science. 251: 1233-1236).

The present demonstration of M-severin induction in transformed epithelial cells not only resolves the apparent paradox of down-regulation of actin filament regulatory proteins in neoplastic cell types, but also provides a natural model system for testing phenotypes resulting from M-severin expression in epithelial cells. Induction of expression of M-severin in normal epithelium and knockout of M-severin in transformed epithelial cells may provide key insights into the functional role of actin filament severing in mammalian cells that has not been possible to define. The human form of M-severin, H-severin has equivalent properties to the mouse M-severin.

Expression of M-severin in LL/2 cells is generalized to other motile mammalian cells and to human carcinoma tumors. In moderately differentiated colon adenocarcinomas, cytoimmunostaining for M-severin is apparent in connective tissue fibroblasts as well as invasive epithelial cells. Western blot and cytoimmunostaining for M-severin has also been obtained from mouse carcinoma tumors, 3T3 fibroblasts, activated lymphocytes and macrophages, leading to our hypothesis that actin cytoskeleton proteins dedicated to motility and cytokinesis are specifically expressed during epithelial cell transformation and leucocyte activation. Messenger RNA expression patterns of M-severin during transformation further demonstrate a correlation between M-severin expression and progressive metastatic potential of epithelial cell lines. Cloning of the full-length cDNA will be required for unequivocal definition of the function of M-severin in mammalian cells and its role in the acquisition of motility during epithelial cell transformation.

This work provides the initial observation of the replacement of an actin regulatory protein in sessile epithelial cells with one of similar function from a highly motile cell type. We posit that alternate cytoskeletal gene expression may constitute a general biological mechanism for enhancing the migratory and proliferative potential of transformed epithelium and leucocytes. This hypothesis is lent credence by our observation that M-severin becomes selectively expressed in transformed, invasive epithelium in adenocarcinomas of the colon.

2. M-30 Localization

Herein it is shown that M-30 protein is the mammalian homolog of Dictyostelium 30 kDa bundling protein that is abundantly expressed at sites of actin cytoskeleton rearrangements in normal migratory cells and neoplastic epithelium. Expression of this actin cytoskeleton regulatory protein coincides with a gain of motility function in subconfluent MDCK epithelial cells, dividing and migrating border cells of wounded epithelium, transformed epithelial LL/2 cells, murine Lewis lung carcinoma tumors undergoing cell migration and transformed epithelial mucosal cells in human colon adenocarcinoma.

Several conclusions from this study are noteworthy. First, at least two new cytoskeletal regulators, M-30 and M-severin, become expressed during transition from the non-motile phenotype found in epithelial sheets to unattached and freely motile cells. Second, motility is an inherent property of epithelial cells in monolayers, requiring only release from the restraints of cell to cell attachment to be induced. In this case cell-cell detachment apparently triggers exogenous induction of an actin regulatory protein (M-30) in a process designed to generate cell motility. Actin regulators other than M-30 and M-severin may also be involved in this process. Third, signal transduction through E-cadherin may be directly coupled to transcriptional regulation of cytoskeletal genes. Such a nexus between disruption of cell-cell contacts and actin cytoskeleton transcription has not been described, but is clearly implicated by the present observations of M-30 expression in cells with disrupted intercellular contacts bordering wounds and by down-regulation of M-30 as cell contacts are made during monolayer formation.

E-cadherin binding has been established as the prime initial event in signal transduction events emanating from epithelial cell-cell attachment, because of its identification as the zonula adherens cell-cell adhesion receptor, and because loss of E-cadherin function results in immediate loss of junctions and transformation of quiescent epithelial layer cells into a highly motile phenotype, (Gumbiner and Simons, 1986; Nagafuchi, et al., 1987; Matsuzaki, et al., 1990; McNeill et al., 1990; reviewed in Takeichi, 1991). Herein is provided a mechanism for controlling cytoskeleton rearrangement by regulated expression of cytoskeletal proteins in response to gain or loss of cell adherence. With the report of specific expression of motility regulatory proteins such as M-30 associated with the breaking of cell contacts, it is possible to follow transcriptional events giving rise to specific cytoskeletal changes leading to motile epithelial phenotypes.

A striking feature of M-30 behavior during transition to monolayers was its relatively rapid degradation. Normal turnover times for actin cytoskeleton components are quite slow, ranging from several days for tropomyosin and troponins to months for myosin and actin. Significant decreases in M-30 presence were noted only hours after cells had initially clustered into monolayer islets.

Transitions between normal and transformed epithelial phenotypes are clearly accompanied by extensive rearrangements of the actin cytoskeleton. Assembly of the dynamic actin filament complexes found at the leading edge of motile cells has proven to be an extraordinarily complicated process, involving a large number of actin-associated proteins and regulators (reviewed in Luna and Hitt, 1992). Regulated assembly, disassembly, cross-linking and membrane attachment of actin filaments are the dominant processes required for extension of the cell margin (reviewed by Pollard and Cooper; Stossel, et al.; Luna and Hitt; Warwick and Spudich). However, none of the in vitro properties suggest expression regulated by cell-cell contacts. Moreover, the pronounced expression of M-30 Protein in transformed epithelial cells and human colon adenocarcinoma tumors indicates a surprising use as an early transformation marker for carcinomas. Also, the mammalian homolog of Dictyostelium severin (M-severin) is heavily expressed in transformed epithelial LL/2 cells, coincident with the extensive down-regulation of gelsolin, the prevalent F-actin severing protein in confluent epithelial cell sheets. Here again, an actin regulatory protein from a primitive organism specialized for optimum cell migration is utilized during the conversion of sessile cells in epithelia to a state of active proliferation and motility.

Those of skill in the art will recognize the utility and the scope of the invention herein described and its applications in qualitative assays, quantitative assays and screening assays (including high throughput, mass screening and small scale or individual assays). One of skill in the art will also recognize the uses of the present invention in the identification and development of novel drug candidates for the acceleration of wound healing and the management or treatment of disorders of the regulation cytoskeletal structures within the cell including those which lead to neoplastic disease.

3 Tyrosine Phosphorylation of M-30

The present inventors have used confocal microscopy to provide new information on the location of M30 protein and Annexin II in various compartments of dividing and motile transformed epithelial cells. Antibodies selective for M30, Annexin II, phosphotyrosine, or actin were used to identify and localize LLC proteins. In addition, selective biochemical extractions of cells showed exclusive segregation of these proteins to either the membrane or the internal actin network. M30 is further shown to be the dominant tyrosine phosphorylated protein in LL2 cells. M30 relocates from adhesion plaques on the cell floor to the nascent cleavage furrow during cell division, identifying a new component of the cleavage furrow and focal contact sites. Annexin II is also identified as a new component of transformed epithelial cells which is associated with the cell membrane.

The following patents and scientific publications may be useful in practicing the fall scope of the invention. These patents are incorporated herein by reference in their entirety. The scientific literature is cited to give an indication of the available art known to the skilled artisan in the field. These patents and publications are provided for illustrative purposes and should not be construed as limiting in any way.

U.S. Pat. No. 5,374,544 is entitled “Mutated skeletal actin promoter.” U.S. Pat. No. 5,464,817 is entitled “Methods for reducing the viscosity of pathological mucoid airway contents in the respiratory tract comprising addministering actin-binding compounds with or without DNAse I.”

U.S. Pat. No. 5,508,265 entitled “Therapeutic uses of actin-binding compounds” discloses the use of actin-binding compounds, including gelsolin and biologically active fragments thereof in the treatment of actin-related disorders. U.S. Pat. No. 5,593,964 is entitled “Methods of treating septic shock by preventing actin polymerization.”

U.S. Pat. No. 5,656,589 is entitled “Method for the reduction of viscous purulent airway contents in the respiratory tract comprising administering actin-binding compounds with or without DNAse I.” U.S. Pat. No. 5,851,786 is entitled “Product and process to regulate actin polymerization.”

U.S. Pat. No. 5,071,773 entitled “Hormone receptor-related bioassays” discloses assay methods using transcriptional reporter genes generally useful for high throughput screening. Such screens may be adapted for use of assays employing genes encoding actin-binding and regulatory proteins in addition to the steroid hormone receptors which act as transcription factors. U.S. Pat. No. 5,401,629 discloses further screening methods using readouts based on detecting changes in the transcription of reporter genes engineered to express a detectable signal in response to activation by intracellular signaling pathways. The transcriptional assay methods exemplified in these two US patents are of particular utility in the screening methods of the present invention and are incorporated herein by reference.

U.S. Pat. No. 5,482,835 entitled “Methods of Testing in Yeast Cells for Agonists and Antagonists of Mammal G protein-Coupled Receptors” discloses methods for screening; U.S. Pat. No. 5,747,267 also discloses yeast screens and is entitled “Method for Identifying a G Protein-Coupled Glutamate Receptor Agonist and Antagonist”; The transcriptional assay methods exemplified in these two US patents are of particular utility in the screening methods of the present invention and are incorporated herein by reference.

U.S. Pat. No. 5,750,353 entitled “Assay for Non-peptide Agonists to Peptide Hormone Receptors” discloses further screening methods; as does U.S. Pat. No. 5,925,529 entitled “Method for Discovery of Peptide Agonists”; U.S. Pat. No. 5,744,303 is entitled “Functional Assays for Transcriptional Regulator genes”; and U.S. Pat. No. 5,569,588 discloses “Methods for Drug Screening”. The transcriptional assay methods exemplified in these four US patents are of particular utility in the screening methods of the present invention and are incorporated herein by reference.

Andre, E. A., M. Brink, G. Gerisch, G. Isenberg, A. Noegel, M. Schleicher, J. E. Segall, and E. Wallraff. 1989. J. Cell Biol. 108: 985-995. Is entitled: “A Dictyostelium mutant deficient in severin, an F-actin fragmenting protein, shows normal motility and chemotaxis”. Yin, H. L. et al. 1990. FEBS LETT. 264(1): 78-80 is entitled “Severin is a gelsolin phenotype”.

Jones, J. G., J. Segal and J. Condeelis. 1991. Experientia-Suppl. 59: 1-16 is entitled “Molecular analysis of amoeboid chemotaxis: parallel observations in amoeboid phagacytes and metastatic tumor cells.” Eichinger et al. 1991. J. Cell. Biol. 112(4): 665-76 is entitled “Domain structure in actin-binding proteins: expression and functional characterization of truncated severin.” Prendergast, G. C. and E. B. Ziff 1991. EMBO J. 10(4): 757-66 is entitled “Mbh1: a novel gelsolin/severin-related protein which binds actin in vitro and exibits nuclear localization in vivo.”

Finidori et al. 1992. J. Cell. Biol. 116(5): 1145-55 is entitled “In vivo analysis of functional domains from villin and gelsolin.” Eichinger, L. and M. Schleicher. 1992. Biochemistry 31(20) 4779-87 is entitled “Characterization of actin- and lipid-binding domains in severin, a Ca(2+)-dependent F-actin fragmenting protein.”

Schnuchel et al. 1995. J. Mol. Biol. 247(1): 21-7 is entitled “Structure of severin domain 2 in solution.” Folger, P. A. 1996. Ph.D. thesis, Cornell University, entitled “Identification, isolation and expression of M-severin, a novel actin filament severing protein in murine carcinoma tumors.”

Markus et al. 1997. Protein Sci. 6(6): 1197-1209 is entitled “Refined structure of villin 14T and a detailed comparison with other actin-severing domains.” Eichinger, L. et al. 1998. J. Biol. Chem. 273(21): 12952-9 is entitled “Characterization and cloning of a Dictyostelium Ste20-like protein kinase that phosphorylates the actin-binding protein severin.” And Weber, I., Niewohner, J., and Faix, J. 1999. Biochem. Soc. Symp. 65:245-65 is entitled “Cytoskeletal protein mutations and cell motility in Dictyostelium.”

1. An isolated actin-binding regulatory protein which (i) is expressed in motile, proliferating, and invasive cells, and in cells at the site of a wound, and (ii) is up-regulated in tumor cells.

2. The isolated actin-binding regulatory protein of claim 1 which protein is H-severin.

3. The isolated actin-binding regulatory protein of claim 1 which protein is H-30.

4. The isolated protein of claim 1 capable of severing F-actin filaments.

5. A nucleic acid molecule encoding an actin-binding regulatory protein of claim 1.

6. The nucleic acid molecule of claim 5 encoding H-severin protein.

7. The nucleic acid molecule of claim 5 encoding H-30 protein.

8. A nucleic acid vector comprising a nucleic acid molecule of claim 5.

9. A host cell comprising the nucleic acid vector of claim 8.

10. An antibody which specifically binds an epitope of a human actin-binding regulatory protein which (i) is expressed in motile, proliferating, and invasive cells, and in cells at the site of a wound, and (ii) is up-regulated in tumor cells.

11. The antibody of claim 10 which specifically binds an epitope of H-severin.

12. The antibody of claim 10 which specifically binds an epitope of H-30.

13. The antibody of claim 10 which comprises a monoclonal antibody.

14. A method of determining the proliferative status or stage of carcinogenesis of a human cell, comprising:
(i) providing a cell sample, and
(ii) assessing the levels of expression or activity of a human actin-regulatory protein in at least one cell of the cell sample, wherein a high level of expression or activity of the human actin-regulatory protein correlates with cellular proliferation, motility, neoplasia or wound healing, thereby determining the proliferative status or stage of carcinogenesis of the cell.

15. The method according to claim 14 wherein the proliferative status or stage of carcinogenesis is a precancerous condition.

16. The method according to claim 14 in which the level of human actin-regulatory protein is assessed by a northern blot, western blot or cytoimmunohistochemistry.

17. A method of identifying a test compound as a modulator of expression or activity of an actin-regulatory protein, comprising:
(i) providing a first cell expressing the actin-regulatory protein,
(ii) contacting the first cell with the test compound,
(iii) assessing the level of expression or activity of the actin-regulatory protein in the first cell,
(iv) providing a second cell identical to the first cell expressing actin-regulatory protein and having actin-regulatory protein activity, which has not been contacted with the test compound,
(v) assessing the expression or activity of the actin-regulatory protein in the second cell, and
(vi) comparing the expression or the activity of actin-regulatory protein in
the first and second cell, thereby determining whether the test compound is a modulator of actin-regulatory protein expression or activity.

18. The method of claim 17 wherein the actin-regulatory protein is H-severin.

19. The method of claim 17 wherein the actin-regulatory protein is H-30.

20. A method of inhibiting proliferation of a human cell in a stage of carcinogenensis comprising administering to the cell an effective amount of a compound which inhibits human actin-binding regulatory protein expression or activity such that the carcinogenesis is inhibited.

21. The method of claim 20 wherein the human actin-binding regulatory protein is H-severin or H-30.

22. The method of claim 20 wherein the human cell is present in a human being.

23. The method of claim 20 wherein the compound inhibits the transcription of the human actin-binding regulatory protein.

24. The method of claim 20 wherein the compound inhibits the translation or post-translational modification of the human actin-binding regulatory protein.

25. The method of claim 20 wherein the compound inhibits the actin-binding or actin severing activity of the human actin-binding regulatory protein.
